(19) United States
(12) Patent Application Publication
(10) Pub. No.: US 2002/0082411 A1
(43) Pub. Date: Jun. 27, 2002

(54) Immune mediators and related methods

(76) Inventors: Darrick Carter, Seattle, WA (US); Shirley Zhu, San Mateo, CA (US); Subhashini Arimilli, Fremont, CA (US); Aijun Wang, Issaquah, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/815,837

(22) Filed: Mar. 22, 2001

Related U.S. Application Data
(60) Provisional application No. 60/264,003, filed on Jan. 23, 2001.

(51) Int. Cl. C07H 21/04; C12P 21/04; C12N 5/06; C07K 14/74

(52) U.S. Cl. 536/23.5; 530/351; 435/69.7; 435/320.1; 435/325

(57) ABSTRACT

The present invention relates to nucleic acids encoding single chain MHC class II molecules that form multimers via inter-chain multimerization domains, and methods of treating autoimmune disease using the same.

### CROSS-REFERENCES TO RELATED APPLICATIONS

This application is related to U.S. Ser. No. 60/191,274, filed Mar. 22, 2000; U.S. Ser. No. 60/204,249, filed May 15, 2000; and U.S. Ser. No. 60/264,003, filed Jan. 23, 2001. Each of the aforementioned applications are herein incorporated by reference in their entirety.

This application is also related to U.S. Ser. No. 09/261,811, filed Mar. 3, 1999; which is a continuation of U.S. Ser. No. 08/657,581, filed Jun. 7, 1996, now abandoned; which is a continuation in part of U.S. Ser. No. 08/480,002, filed Jun. 7, 1995, now abandoned; U.S. Ser. No 09/184,692, filed Nov. 2, 1998, now abandoned; U.S. Ser. No. 08/483,241, filed Jun. 7, 1995; U.S. Ser. No. 08/482,133, filed Jun. 7, 1995; and U.S. Provisional Application No. 60/005,964, filed Oct. 27, 1995.

### BACKGROUND OF THE INVENTION

T cells, unlike B cells, do not directly recognize antigens. Instead, an accessory cell must first process an antigen and present it in association with an MHC molecule in order to elicit a T cell-mediated immunological response. The major function of MHC glycoproteins appears to be the binding and presentation of processed antigen in the form of short antigenic peptides.

In addition to binding foreign or “non-self antigenic peptides,” MHC molecules can also bind “self” peptides. If T lymphocytes then respond to cells presenting “self” or autoantigenic peptides, a condition of autoimmunity results. Over 30 autoimmune diseases are presently known, including myasthenia gravis (MG), multiple sclerosis (MS), systemic lupus erythematosus (SLE), rheumatoid arthritis (RA), insulin-dependent diabetes mellitus (IDDM), etc. Characteristic of these diseases is an attack by the immune system on the tissues of the host. In non-diseased individuals, such attack does not occur because the immune system recognizes these tissues as “self.” Autoimmunity occurs when a specific adaptive immune response is mounted against self tissue antigens.

There is therefore currently a great interest in developing pharmaceuticals based on the growing understanding of the structure and function of the major histocompatibility complex (MHC) antigens. Identification of synthetic autoantigenic peptides, and demonstration that these peptides bind selectively to MHC molecules associated with disease and that stimulates T cells would help to implicate a particular peptide or peptide:MHC complex in susceptibility to an autoimmune disease. In particular, the development of single chain MHC class II complexes would be particularly useful in treatment of a number of diseases associated with antigen presentation by MHC molecules. Furthermore, the development of single chain, multimeric complexes would be of interest (see, e.g., WO 93/10220, WO 98/05684, WO 97/35991, WO 98/03552, WO 99/13095, WO 98/06749, WO 99/09064, and U.S. Pat. No. 5,869,270).

### SUMMARY OF THE INVENTION

The present invention provides recombinant nucleic acid constructs that encode single chain, recombinant MHC class II molecules comprising a β1 domain and an α1 domain that may or may not be further linked to an antigenic peptide. In one embodiment, the single chain polypeptide is a β1 domain and an α1 domain. In another embodiment, the single chain polypeptide is a β1 domain-β2 domain (a β chain) and an α1 domain-α2 domain (an α chain). The single chain constructs of the invention can be further dimerized or multimerized by inter-chain fusion. The fusion sequence (also referred to as a dimerization or multimerization sequence) can be any sequence that allows for covalent or non-covalent linkages between the molecules of the invention. As shown below, a preferred means for carrying this out is through use of segments from immunoglobulin family proteins (e.g., antibodies, MHC molecules, T cell receptors and the like) that have cysteine residues capable of forming interchain disulfide bonds (e.g., constant regions from Ig light chains, e.g. Cκ or Cλ, or constant regions from Ig heavy chains, e.g., CH1, hinge, CH2, or CH3). In another embodiment, a leucine zipper domain forms a non-covalent linkage. One of skill will recognize that any of a number of polypeptide sequences can be used for this purpose. The single chain molecules of the invention thus can be multimers wherein each single chain molecule is from a different MHC class II allele. In addition, each single chain molecule in the multimer can be bound to a different antigen.

In one embodiment, monomeric and dimeric forms of recombinant single chain mouse I-AS-peptide complexes, fused to an antigenic MBP 90-101 peptide with flexible linkers were constructed. The recombinant single chain I-AS proteins share structural similarity to that of crystallized native human MHC class II protein as determined by protein modeling. The recombinant single chain proteins were expressed in E. coli and in an insect expression system and purified by affinity chromatography and FPLC. The purified single chain recombinant I-AS proteins showed in vitro biological activity as assayed using an antigen-specific mouse T cell clone. The in vivo activity of the recombinant single chain I-AS fusion proteins in the EAE model using susceptible SJL mice shows that treatment with the recombinant single chain I-AS proteins prevents mortality and significantly reduces paralysis induced by myelin homogenate. Histological examination of sections from animal spinal cord reveals that these treatments also reduce the inflammatory lesions. These results demonstrate that the single chain MHC class II molecules have therapeutic benefit as antigen-specific drugs for the treatment of autoimmune diseases.

In another embodiment, novel linkers are provided for forming single chain MHC class II molecules. These linkers can be used with the multimer constructs described above. In another embodiment, the constructs of the invention are optimized for prokaryotic expression, using codons adjusted for E. coli codon bias.

The present invention also provides MHC class II heterodimers, wherein a recombinant β chain and a recombinant α chain are covalently linked using polypeptide fusion segments, e.g., from immunoglobulin family proteins (e.g., antibodies, MHC molecules, T cell receptors and the like) that have cysteine residues capable of forming interchain disulfide bonds (e.g., constant regions from Ig light chains, e.g. Cκ or Cλ, or constant regions from Ig heavy chains, e.g., CH1, hinge, CH2, or CH3). Such a heterodimer can also be “dimerized” or “multimerized” by the use of additional fusion domains, such as leucine zipper domains or immunoglobulin domains (see FIG. 10).

### BRIEF DESCRIPTION OF THE DRAWINGS

FIG. 1. Schematic structure of the recombinant single chain I-AS .MBP.β1α1 (monomer) and I-AS.MBP.β1β2α1α2.CK (dimer) proteins.

FIG. 2. In vitro biological activities of the recombinant I-AS proteins compared with APC+antigenic peptide in the mouse T cell clone, HS-1. A. I-AS.MBP.β1α1, monomer, B. I-AS.MBP.Ck, dimer, and C. APC+ antigenic peptide. The positive (anti-CD3) and the negative (HS-1 cell alone) controls are also shown in each panel.

FIG. 3. Diagram of the EAE model and standard for EAE scoring.

FIG. 4. The EAE model studies. The recombinant I-AS proteins were administered to SJL mice on day 1, 4, 7, and II by i.v. injection after inducing the disease with myelin emulsified in CFA. The animals were evaluated for neurological dysfunction. Panel A: Untreated, injected with same amount of PBS solution. Panel B. Treated with the recombinant I-AS.MBP.Ck protein, a dimer form. Panel C: Treated with the recombinant I-AS.β1α1. This recombinant molecule does not carry the antigenic peptide. Panel D: Treated with the recombinant I-AS.MBP.β1α1, a monomer form.

FIG. 5 shows a schematic representation of a β1-α1 single chain MHC class II peptide complex that is a dimer with two peptide specificities.

FIG. 6 shows a schematic representation of a β1β2-α1α2 single chain MHC class II peptide complex that is a dimer with two peptide specificities.

FIG. 7 shows a schematic representation of a β1β2-α1α2 single chain MHC class II peptide complex that is a tetramer with four peptide specificities.

FIG. 8 shows a schematic representation of a β1β2-α1α2 single chain MHC class II peptide complex that is a tetramer with two peptide specificities and two different MHC class II alleles.

FIG. 9 shows the effect of different recombinant MHC class II molecules on the development of EAD (day 60+).

FIG. 10. Diagram of β1-α1 single chain MHC class II peptide complex; diagram of recombinant β1β2 chains fused to recombinant α1α2 chains via a fusion domain from an immunoglobulin; and diagram of multimerized MHC class II molecules.

FIG. 11. Sequence comparison of mouse CO608 single chain molecules.

FIG. 12. Sequence comparison of mouse CO561 single chain molecules.

### DETAILED DESCRIPTION OF THE INVENTION

### Introduction

The present invention provides recombinant DNA constructs that encode single chain MHC class II molecules that may or may not be further linked to an antigenic peptide. Typically, the constructs comprise a first DNA segment encoding a β1 domain of a selected MHC class II molecule; a second DNA segment encoding a α1 domain of the selected MHC class II molecule; and a first linker DNA segment connecting in-frame the first and second DNA segments; wherein linkage of the first DNA segment to the second DNA segment by the first linker DNA segment results in a fused first DNA-first linker-second DNA polysegment. The constructs of the invention may also comprise a third DNA segment encoding an antigenic peptide capable of associating with a peptide binding groove of the selected MHC class II molecule and a second linker DNA segment connecting in-frame the third DNA segment to the fused first DNA-first linker-second DNA polysegment.

In another embodiment, the present invention also provides recombinant components of an MHC class II heterodimer, which comprise a fusion domain. One recombinant component comprises a β1 domain, or optionally a β1 domain-β2 domain (i.e., β chain). One recombinant component comprises an α1 domain, or optionally an α1 domain-α2 domain (i.e., an α chain). The two recombinant chains are linked, either covalently, e.g., via a disulfide bond, or non covalently, using the fusion domain. Such molecules can also be made into multimers using additional fusion or multimerization domains. In one embodiment, the invention provides the following recombinant components of an MHC class II heterodimer: pCB220, which is an IAS.MBP.alpha chain fused to an IgG2a CH1 and truncated hinge region; pCB223, which is an IAS.MBP.alpha chain fused to an IgG2a CH1.H.CH2.CH3; and pCB229, which is an IAS.MBP.beta chain fused to a mouse Cκ domain. These recombinant components can be fused via the fusion domain to form a MHC class II heterodimer molecule that is covalently linked via a disulfide bond at the fusion domain.

The present invention provides single chain MHC class II molecules that comprise an additional polypeptide sequence that allows for inter-chain dimerization of the single chain molecules of the invention. The additional polypeptide allows multimerization of the single chain MHC class II molecules, to produce, e.g., dimers and tetramers. The sequence can be any sequence that allows for covalent or non-covalent linkages between the molecules of the invention. In one embodiment, the single chain molecules are covalently linked using chemical methods known to those of skill in the art, e.g., photoaffinity methods or homo-bifunctional protein cross-linkers (see, e.g., Hermanson et al., Bioconjugate Techniques, (1996)). In one embodiment, the molecules are covalently linked using heterobifunctional protein cross-linkers. As shown below, one means for carrying this out is through use of segments form immunoglobulin family proteins (e.g., antibodies, MHC molecules, T cell receptors and the like) that have cysteine residues capable of forming interchain disulfide bonds. An example shown below is the use of the constant region of the kappa chain of an antibody (Cκ), from either a heavy or a light chain. Other dimerization sequences include a leucine zipper, a STAT protein N-terminal domain, or the FK506 binding protein (see, e.g., O'Shea, Science 254: 539 (1991), Barahmand-Pour et al., Curr. Top. Microbiol. Immunol. 211:121-128 (1996); Klemm et al., Annu. Rev. Immunol. 16:569-592 (1998); Ho et al., Nature 382:822-826 (1996)). One of skill will recognize that any of a number of polypeptide sequences can be used for this purpose.

In addition, the multimeric, single chain class II molecules of the invention comprise at least two different MHC class II alleles that are associated with an autoimmune disease state, and/or at least two different autoantigenic peptides that are associated with a particular autoimmune disease state. In one example, the multimeric, single chain class II molecules have chains from different DR2 alleles, e.g., DRB5*0101 and DRB1*1501. In another embodiment, the autoantigenic peptides are peptides associated with multiple sclerosis, e.g., MBP (e.g., amino acid residues 83-102Y83), PLP (e.g., amino acid residues 40-60, 89-106, 95-117, and 185-206); and MOG. In addition, other antigens associated with autoimmune disease, such as acetylcholine receptor and type II collagen, can be linked to the single chain molecules of the invention.

In a further embodiment, the single chain class II molecules of the invention have novel linkers, as described herein. The mammalian MHC class II single chain constructs of the invention may also be constructed to use preferred prokaryotic codons, for expression, e.g., in E. coli, using codon preference tables and methods known to those of skill in the art.

### Definitions

Prior to setting forth the invention, it may be helpful to an understanding thereof to provide definitions of certain terms to be used hereinafter:

Single chain MHC class II molecule: As used herein this term refers to a fusion protein such as the recombinant single chain MHC class II complex of the invention, which optionally also is fused to a peptide to form a single chain MHC class:peptide complex. The fusion proteins of the invention can also be multimers, having two, four or more single chain molecules linked covalently or non-covalently through multimerization domains in the single chain molecule. A single chain molecule of the invention typically comprises at least an MHC class II β1 domain and an MHC class II α1 domain, optionally β1 β2 α1 α2 domains or any combination thereof in any order. Such molecules are also known as “fused heterodimers.” Optionally, the single chain molecules are soluble, that is, they lack the naturally occurring cytoplasmic and transmembrane MHC class II domains.

A domain of a selected MHC molecule: A portion of an MHC domain which is sufficient to form, either alone, or in combination with another portion of an MHC domain, a peptide binding site which is capable of presenting an antigenic peptide in such a fashion that it is recognized by a T cell receptor. Such MHC domains would include the extracellular portion of the two polypeptide chains of Class II MHC. This would include the α chain (α1 and α2 domains) and β chain (β1 and β2 domains) of Class II MHC. This would include β1 and α1, β1, β2 and α1, α2, α1 or α2 independent of the other, or α1 and α2 in tandem (α1α2). It would also include β1 or β2 independent of the other, or β1 and β2 in tandem (β1β2). This would also include any suitable combination of the α1, α2, β1, and β2 domains. The domains can be directly linked, or can be linked via an amino acid linker.

Linker DNA segment: A segment of DNA encoding from about 1 to about 50, preferably from about 5 to about 25 amino acids, which forms a flexible link between two DNA segments. This flexible link allows the two DNA segments to attain a proper configuration, such as an MHC peptide binding groove, or allows a peptide to properly bind into such a groove.

Antigenic peptide: The immunological properties of MHC histocompatibility proteins are largely defined by the antigenic peptide that is bound to them. An antigenic peptide is one which contains an epitope (an amino acid sequence) recognized by immune cells, e.g., T cells, and is capable of stimulating an MHC-mediated immune response. Antigenic peptides for a number of autoimmune diseases are known. For example, in experimentally induced autoimmune diseases, antigens involved in pathogenesis have been characterized: in arthritis in rat and mouse, native type II collagen is identified in collagen-induced arthritis, and mycobacterial heat shock protein in adjuvant arthritis (Stuart et al., Ann. Rev. Immunol. 2:199-218, 1984; and van Eden et al., Nature 331:171-173, 1988); thyroglobulin has been identified in experimental allergic thyroiditis (EAT) in mice (Marion et al., J. Exp. Med. 152:1115-1120, 1988); acetylcholine receptor (AChR) in experimental allergic myasthenia gravis (EAMG) (Lindstrom et al., Adv. Immunol. 42:233-284, 1988); and myelin basic protein (MBP) and proteolipid protein (PLP) in experimental allergic encephalomyelitis (EAE) in mouse and rat (Acha-Orbea et al., Ann. Rev. Imm. 7:377-405, 1989). In addition, target antigens have been identified in humans: type II collagen in human rheumatoid arthritis (Holoshitz et al., Lancet ii:305-309, 1986), acetylcholine receptor in myasthenia gravis (Lindstrom et al., Adv. Immunol. 42.:233-284, 1988), and MBP, PLP, and MOG in multiple sclerosis in humans.

MHC: The major histocompatibility complex (MHC) is a family of highly polymorphic proteins, divided into two classes, Class I and Class II, which are membrane-associated and present antigen to T lymphocytes (T cells). MHC Class I and Class II molecules are distinguished by the types of cells on which they are expressed, and by the subsets of T cells which recognize them. Class I MHC molecules (e.g., HLA-A, -B and -C molecules in the human system) are expressed on almost all nucleated cells and are recognized by cytotoxic T lymphocytes (CTL), which then destroy the antigen-bearing cells. Class II MHC molecules (HLA-DP, -DQ and -DR, for example, in humans) are expressed primarily on the surface of antigen-presenting cells, such as B lymphocytes, dendritic cells, macrophages, and the like. Class II MHC is recognized by CD4⁺ T helper lymphocytes (T_{H}). T_{H} cells induce proliferation of both B and T lymphocytes, thus amplifying the immune response to the particular antigenic peptide that is displayed (Takahashi, Microbiol. Immunol., 37:1-9, 1993).

Two distinct antigen processing pathways are associated with the two MHC classes. Intracellular antigens, synthesized inside of the cell, such as from viral or newly synthesized cellular proteins, for example, are processed and presented by Class I MHC. Exogenous antigens, taken up by the antigen-presenting cell (APC) from outside of the cell through endocytosis, are processed and presented by Class II MHC. After the antigenic material is proteolytically processed by the MHC-bearing cell, the resulting antigenic peptide forms a complex with the antigen binding groove of the MHC molecule through various noncovalent associations. The MHC-peptide complex on the cell surface is recognized by a specific T cell receptor on a cytotoxic or helper T cell.

The MHC of humans (also referred to as human leukocyte antigens (HLA)) on chromosome 6 has three loci, HLA-A, HLA-B and HLA-C, the first two of which have a large number of alleles encoding alloantigens. An adjacent region, known as HLA-D, is subdivided into HLA-DR, HLA-DQ and HLA-DP. The HLA region is now known as the human MHC region, and is equivalent to the H-2 region in mice. HLA-A, -B and -C resemble mouse H-2K, -D, and -L and are the Class I MHC molecules. HLA-DP, -DQ and -DR resemble mouse I-A and I-E and are the Class II molecules. MHC glycoproteins of both classes have been isolated and characterized (see Fundamental Immunology, 2d Ed., Paul (ed.), (1989); and Roitt et al., Immunology, 2d Ed., (1989), which are both incorporated herein by reference).

Human MHC Class I molecules consist of a polymorphic type I integral membrane glycoprotein heavy chain of about 46 kD, noncovalently associated with a 12 kD soluble subunit, β2-microglobulin. The heavy chain consists of two distinct extracellular regions, the membrane distal, peptide binding region formed by the α1 and α2 domains, and the membrane proximal, CD8-binding region derived from the α3 domain. β₂-microglobulin is a single, compact immunoglobulin-like domain that lacks a membrane anchor, and exists either associated with the class I heavy chain or free in plasma (Germain and Margulies, Annu. Rev. Immunol. 11:403-50, 1993).

Human MHC Class II is a heterodimeric integral membrane protein. Each dimer consists of one α and one β chain in noncovalent association. The two chains are similar to each other, with the α chain having a molecular weight of 32-34 kD and the β chain having a molecular weight of 29-32 kD. Both polypeptide chains contain N-linked oligosaccharide groups and have extracellular amino termini and intracellular carboxy termini.

The extracellular portions of the α and β chain that comprise the class II molecule have been subdivided into two domains of about 90 amino acids each, called α1, α2, and β1, β2, respectively. The α2 and β2 domains each contain a disulfide-linked loop. The peptide-binding region of the class II molecule is formed by the interaction of the α1 and β1 domains. This interaction results in an open-ended, antigenic peptide-binding groove made up of two α helices, and an eight-stranded β-pleated sheet platform.

The α and β chains of Class II molecules are encoded by different MHC genes and are polymorphic (see Addas et a., Cellular and Molecular Immunology, 2d Ed. (1994), which is incorporated by reference in its entirety). Within the present invention, a preferred α chain is DRA*0101 and a preferred β chain is DRβ1*1501.

### MHC Class II Alleles

The single chain MHC class II:peptide complexes of the present invention can incorporate cDNA from any allele that predisposes or increases the likelihood of susceptibility to a specific autoimmune disease. Specific autoimmune diseases are correlated with specific MHC types. Specific haplotypes have been associated with many of the autoimmune diseases. For example, HLA-DR2⁺ and HLA-DR3⁺ individuals are at a higher risk than the general population to develop systemic lupus erythematosus (SLE) (Reinertsen et al., N. Engl. J. Med. 299:515-18, 1970). Myasthenia gravis has been linked to HLA-D (Safwenberg et al., Tissue Antigens 12:136-42,1978. Susceptibility to rheumatoid arthritis is associated with HLA-D/DR in humans. Methods for identifying which alleles, and subsequently which MHC-encoded polypeptides, are associated with an autoimmune disease are known in the art. Exemplary alleles for IDDM include DR4, DQ8, DR3, DQ3.2.

### Uses of Single Chain MHC Class II Molecules

Single chain MHC class II molecules and/or single chain MHC class II:peptide complexes of the present invention can be used as antagonists to therapeutically block the binding of particular T cells and antigen-presenting cells. In addition, the molecules can induce anergy, or proliferative nonreponsiveness, and possibly apoptosis, in targeted T cells, both in vivo and in vitro. A single chain MHC class II:peptide molecule directed toward a desired autoimmune disease contains the antigenic peptide implicated for that autoimmune disease properly positioned in the binding groove of the MHC molecule, without need for solublization of MHC or exogenous loading of an independently manufactured peptide.

Previous methods for producing desirable MHC Class II histocompatibility proteins have provided material that contains a mixture of antigenic peptides (Buus et al., Science 242:1045-1047, 1988; and Rudensky et al., Nature 353:622-627, 1991), which can be only partially loaded with a defined antigenic peptide (Watts and McConnel, Pro. Natl. Acad. Sci. USA 83:9660-64, 1986; and Ceppellini et al., Nature 339:392-94, 1989). Various methods have been developed to produce heterodimers that do not present endogenous antigens (Stem and Wiley, Cell 68:465-77, 1992; Ljunggren et al., Nature 346:476-80, 1990; and Schumacher et al., Cell 62:563-67, 1990) that can be loaded with a peptide of choice. WO 95/23814 and Kozono et al. have described production of soluble murine Class II molecules, I-E^{dk} and I-A^{d}, each with a peptide attached by a linker to the N terminus of the β chain. Ignatowicz et al. (J. Immunol. 154:38-62, 1995) have expressed membrane-bound I-A^{d} with peptide attached. These methods incorporate the use of both membrane-bound heterodimer and soluble heterodimer.

The current invention offers the advantage of a recombinant single chain MHC class II molecule made up of two or more MHC domains joined together via a flexible linkage, and onto which is tethered (via an additional flexible linkage) an antigenic peptide which is able to bind to the peptide binding groove presented by the single chain MHC class II molecule. Such a complex provides an MHC molecule which is soluble and, because the MHC class II components and corresponding antigenic peptide are permanently linked into a single chain configuration, there is no need for complex heterodimer truncation or formation. These complexes eliminate inefficient and nonspecific peptide loading. Producing the claimed MHC:peptide complexes by recombinant methodology results in specific, high yield protein production, where the final product contains only the properly configured MHC:peptide complex of choice.

As used herein, a soluble MHC class II molecules is one that does not contain the naturally occurring membrane-associated MHC class II sequences. The soluble MHC molecules of the present invention has never been membrane-associated. Further, the soluble MHC class II molecules do not contain an amino acid sequence that acts as a transmembrane domain or as a cytoplasmic domain.

The present invention therefore provides a single chain MHC class II molecule which optionally includes an antigenic peptide covalently attached to the amino terminal portion of an α or β chain of MHC through a peptide linkage, and the C terminal of the linked α or β chain may be attached to the N terminal portion of another α or β chain, there by creating a two, three, or four domain MHC molecule. The invention further provides a multimerization domain to provide a multimeric single chain MHC class II molecules. The invention further provides novel linkers, and multimeric MHC class II molecules that are bound to different antigenic peptides.

The amino acid sequence of each of a number of Class I and Class II proteins are known, and the genes or cDNAs have been cloned. Thus, these nucleic acids can be used to express MHC polypeptides. If a desired MHC gene or cDNA is not available, cloning methods known to those skilled in the art may be used to isolate the genes. One such method that can be used is to purify the desired MHC polypeptide, obtain a partial amino acid sequence, synthesize a nucleotide probe based on the amino acid sequence, and use the probe to identify clones that harbor the desired gene from a cDNA or genomic library.

### Linkers

Linkers of the current invention may be from about 1 to about 50 amino acids in length, depending on the molecular model of the MHC or MHC:peptide complex. In one embodiment, flexible linkers are made of repeating Gly residues separated by one or more Ser residues to permit a random, flexible motion. In the case of Class II MHC complexes this flexibility accommodates positioning of the α and β segments to properly configure the binding groove, and also allows for maximum positioning of the peptide in the groove. In another embodiment, the linker comprises a CD4 binding site, as described below in the Example section (see also Table 1). In another embodiment, longer linkers between the chains contain flexible residues (e.g. alanine or glycine) and polar residues (e.g. serine and threonine). To inhibit the continuation of secondary structure across the linker, prolines can be added to bracket the linkers. These prolines are known to inhibit the formation of alpha helices and beta sheets. In another embodiment, flexible regions present in the human MHC and in the murine MHC could be used to make a linker by extending the region of interest and ligating the ends together. Finally, a combination of these types of linkers could also be used.

Linker position and length can be modeled based on the crystal structure of MHC Class II molecules (Brown et al., Nature 364:33-39, 1993), where α1 and β1 are assembled to form the peptide binding groove. Linkers joining segments of the α and β chains together are based on the geometry of the region in the hypothetical binding site and the distance between the C terminus and the N terminus of the relevant segments. Molecular modeling based on the X-ray crystal structure of Class II MHC (Stern et al, Nature 368:215-221, 1994) dictates the length of linkers joining antigenic peptide, α chain segments and β chain segments. The recombinant portions of the molecules of the invention can also be directly linked, without additional amino acids linkers.

### Identification of Autoantigens

The invention also provides methods for preparing responder T-cell clones that proliferate when combined with a selected antigenic peptide presented by a stimulator cell. Such clones can be used to identify and map antigenic peptides associated with autoimmune disease. These peptides can then be incorporated into the single chain MHC class II molecule:peptide complexes of the invention. The method provides isolation and enrichment of non-adherent, CD56⁻, CD8⁻ T cells that are reactive with a selected antigenic peptide. These cells are herein referred to as responder cells. Suitable responder cells can be isolated, for example, from peripheral blood mononuclear cells (PBMNC) obtained from patients prior to or after onset of an autoimmune disease of interest. For example, PBMNCs can be obtained from prediabetic and new onset diabetic patients. These patients can be pre-screened for specific HLA markers, such as DR3-DR4 or DQ3.2, which have the highest association with susceptibility to IDDM. From the collected PBMNCs, a portion is kept to serve as stimulator cells. From the remainder, the desired autoreactive responder cells are purified and isolated by two rounds of plating, to remove adherent cells from the population, followed by removal of monocytes and B cells with nylon wool. Enrichment for non-adherent CD4⁺ T cells is completed by sequential plating of the cells onto plates coated with anti-CD8 and anti-CDS6 antibodies.

The stimulator cells are pulsed or primed with whole GAD or an appropriate antigenic peptide. For example, stimulator cells from the PBMNCs of IDDM patients can be stimulated with antigenic GAD peptides then combined with PBMNCs or responder cells. After seven or 14 days, responder cell (T cell) clones are generated through limiting dilution and tested for antigen reactivity.

These responder cell (T cell) clones can then be used, for example, to map epitopes which bind to MHC and are recognized by a particular T cell. One such method uses overlapping peptide fragments of the autoantigen which are generated by tryptic digestion, or more preferably, overlapping peptides are synthesized using known peptide synthesis techniques. The peptide fragments are then tested for their ability to stimulate the responder T cell clones or lines (for example, Ota et al., Nature 346:183-187, 1990).

Once such a peptide fragment has been identified, synthetic antigenic peptides can be specifically designed, for example, to enhance the binding affinity for MHC and to out-compete any naturally processed peptides. Such synthetic peptides, when combined into a single chain MHC class II molecule:peptide complex, would allow manipulation of the immune system in vivo, in order to tolerize or anergize disease-associated activated T cells, thereby ameliorating the autoimmune disease.

Dissecting the functional role of individual peptides and peptide clusters in the interaction of a peptide ligand with an MHC molecule, and also in subsequent T cell recognition and reactivity, is a difficult undertaking due to the degeneracy of peptide binding to the MHC. Changes in T cell recognition or in the ability of an altered peptide to associate with MHC can be used to establish that a particular amino acid or group of amino acids comprises part of an MHC or T cell determinant. The interactions of altered peptides can be further assessed by competition with the parental peptide for presentation to a T cell, or through development of direct peptide-MHC binding assays. Changes to a peptide that do not involve MHC binding could well affect T cell recognition. For example, in a peptide, specific MHC contact points might only occur within a central core of a few consecutive or individual amino acids, whereas those amino acids involved in T cell recognition may include a completely different subset of residues.

In a preferred method, residues that alter T cell recognition are determined by substituting amino acids for each position in the peptide in question, and by assessing whether such change in residues alters the peptide's ability to associate with MHC (Allen et al., Nature 327:713-15, 1987; Sette et al., Nature 328:395-99, 1987; O'Sullivan et al., J. Immunol. 147:2663-69, 1991; Evavold et al., J. Immunol. 148:347-53, 1992; Jorgensen et al., Annu. Rev. Immunol. 10:835-73, 1992; Hammer et al., Cell 74:197-203, 1993; Evavold et al., Immunol. Today 14:602-9, 1993; Hammer et al., Proc. Natl. Acad. Sci. USA 91:4456-60, 1994; and Reich et al., J. Immunol 154:2279-88, 1994). One method would involve generating a panel of altered peptides wherein individual or groups of amino acid residues are substituted with conservative, semi-conservative or non-conservative residues. A preferred variant of this method is an alanine scan (Ala scan) where a series of synthetic peptides are synthesized wherein each individual amino acid is substituted with L-alanine (L-Ala scan). Alanine is the amino acid of choice because it is found in all positions (buried and exposed), in secondary structure, it does not impose steric hindrances, or add additional hydrogen bonds or hydrophobic side chains. Alanine substitutions can be done independently or in clusters depending on the information desired. Where the information pertains to specific residues involved in binding, each residue in the peptide under investigation can be converted to alanine and the binding affinity compared to the unsubstituted peptide. Additional structural and conformational information regarding each residue and the peptide as a whole can be gained, for example, by synthesizing a series of analogs wherein each residue is substituted with a D-amino acid such as D-alanine (D-Ala scan) (Galantino et al., in Smith, J. and Rivier, J. (eds.), Peptides Chemistry and Biology (Proceedings of the Twelfth American Peptide Symposium), ESCOM, Leiden, 1992, pp. 404-05). Essential residues can be identified, and nonessential residues targeted for modification, deletion or replacement by other residues that may enhance a desired quality (Cunningham and Wells, Science 244:1081-1085, 1989; Cunningham and Wells, Natl. Acad. Sci. USA, 88:3407-3411, 1991; Ehrlich et al., J. Biol. Chem. 267:11606-11, 1992; Zhang et al., Proc. Natl. Acad. Sci. USA 90:4446-50, 1993; see also “Molecular Design and Modeling: Concepts and Applications Part A Proteins, Peptides, and Enzymes,” Methods in Enzymology, Vol. 202, Langone (ed.), Academic Press, San Diego, Calif., 1991).

Truncated peptides can be generated from the altered or unaltered peptides by synthesizing peptides wherein amino acid residues are truncated from the N- or C-terminus to determine the shortest active peptide, or between the N- and C-terminus to determine the shortest active sequence. Such peptides could be specifically developed to stimulate a response when joined to a particular MHC to form a peptide ligand to induce anergy in appropriate T cells in vivo or in vitro.

### Analysis of Single Chain MHC Class II Molecule:peptide Complexes

The physical and biological properties of the single chain MHC class II molecule:peptide complexes may be assessed in a number of ways. Mass spectral analysis methods such as electrospray and Matrix-Assisted Laser Desorption/Ionization Time Of Flight mass spectrometry (MALDI TOF) analysis are routinely used in the art to provide such information as molecular weight and confirm disulfide bond formation. FACs analysis can be used to determine proper folding of the single chain complex.

An ELISA (Enzyme-linked Immunosorbent Assay) can be used to measure concentration and confirm correct folding of the single chain MHC class II molecule:peptide complexes. This assay can be used with either whole cells; solubilized MHC, removed from the cell surface; or free single chain MHC class II molecule:peptide complexes of the current invention. In an exemplary ELISA, an antibody that detects the recombinant MHC haplotype is coated onto wells of a microtiter plate. In a preferred embodiment, the antibody is L243, a monoclonal antibody that recognizes only correctly folded HLA-DR MHC dimers. One of skill in the art will recognize that other MHC Class II-specific antibodies are known and available. Alternatively, there are numerous routine techniques and methodologies in the field for producing antibodies (for example, Hurrell, (ed)., Monoclonal Hybridoma Antibodies: Techniques and Applications, CRC Press Inc., Boca Raton, Fla., 1982), if an appropriate antibody for a particular haplotype does not exist. Anti-MHC Class II antibodies can also be used to purify Class II molecules through techniques such as affinity chromatography, or as a marker reagent to detect the presence of Class II molecules on cells or in solution. Such antibodies are also useful for Western analysis or immunoblotting, particularly of purified cell-secreted material. Polyclonal, affinity purified polyclonal, monoclonal and single chain antibodies are suitable for use in this regard. In addition, proteolytic and recombinant fragments and epitope binding domains can be used herein. Chimeric, humanized, veneered, CDR-replaced, reshaped or other recombinant whole or partial antibodies are also suitable.

In the ELISA format, bound MHC molecules can be detected using an antibody or other binding moiety capable of binding MHC molecules. This binding moiety or antibody may be tagged with a detectable label, or may be detected using a detectably labeled secondary antibody or binding reagent. Detectable labels or tags are known in the art, and include fluorescent, colorimetric and radiolabels, for instance.

Other assay strategies can incorporate specific T-cell receptors to screen for their corresponding MHC-peptide complexes, which can be done either in vitro or in vivo. For example, an in vitro anergy assay determines if non-responsiveness has been induced in the T cells being tested. Briefly, an MHC molecule containing antigenic peptide in the peptide binding groove can be mixed with responder cells, preferably peripheral blood mononuclear cells (PBMN) (a heterogeneous population including B and T lymphocytes, monocytes and dendritic cells), PBMNC lymphocytes, freshly isolated T lymphocytes, in vivo primed splenocytes, cultured T cells, or established T cell lines or clones. Responder cells from mammals immunized with, or having a demonstrable cellular immune response to, the antigenic peptide are particularly preferred.

Subsequently, these responder cells are combined with stimulator cells (antigen presenting cells; APCs) that have been pulsed or primed with the same antigenic peptide. In a preferred embodiment, the stimulator cells are antigenic peptide-presenting cells, such as PBMNCs, PBMNCs that have been depleted of lymphocytes, appropriate antigenic peptide-presenting cell lines or clones (such as EBV-transformed B cells), EBV transformed autologous and non-autologous PMNCs, genetically engineered antigen presenting cells, such as mouse L cells or bare lymphocyte cells BLS-1, in particular, DRB1*0401, DRB1*0404 and DRB1*0301 (Kovats et al., J. Exp. Med. 179:2017-22, 1994), or in vivo or in vitro primed or pulsed splenocytes. Stimulator cells from mammals immunized with, or having a demonstrable cellular immune response to, the antigenic peptide are particularly preferred. For certain assay formats, it is preferred to inhibit the proliferation of stimulator cells prior to mixing with responder cells. This inhibition may be achieved by exposure to gamma irradiation or to an anti-mitotic agent, such as mitomycin C, for instance. Appropriate negative controls are also included (nothing; syngeneic APC; experimental peptide; APC+Peptide; MHC:peptide complex; control peptide+/−APC). Further, to assure that non-responsiveness represents anergy, the proliferation assay may be set up in duplicate, +/−recombinant IL-2 since it has been demonstrated that IL-2, can rescue anergized cells.

After an approximately 72 hour incubation, the activation of responder cells in response to the stimulator cells is measured. In a preferred embodiment, responder cell activation is determined by measuring proliferation using ³H-thymidine uptake (Crowley et al., J. Immunol. Meth. 133:55-66, 1990). Alternatively, responder cell activation can be measured by the production of cytokines, such as IL-2, or by determining the presence of responder cell-specific, and particularly T cell-specific, activation markers. Cytokine production can be assayed by testing the ability of the stimulator+responder cell culture supernatant to stimulate growth of cytokine-dependent cells. Responder cell- or T cell-specific activation markers may be detected using antibodies specific for such markers.

Preferably, the single chain MHC class II molecule:peptide complex induces non-responsiveness (for example, anergy) in the antigenic peptide-reactive responder cells. In addition to single chain MHC class II molecule:peptide complex recognition, responder cell activation requires the involvement of co-receptors on the stimulator cell (the APC) that have been stimulated with co-stimulatory molecules. By blocking or eliminating stimulation of such co-receptors (for instance, by exposing responder cells to purified single chain MHC class II molecule:peptide complex, by blocking with anti-receptor or anti-ligand antibodies, or by “knocking out” the gene(s) encoding such receptors), responder cells can be rendered non-responsive to antigen or to single chain MHC class II molecule:peptide complex.

In a preferred embodiment, responder cells are obtained from a source manifesting an autoimmune disease or syndrome. Alternatively, autoantigen-reactive T cell clones or lines are preferred responder cells. In another preferred embodiment, stimulator cells are obtained from a source manifesting an autoimmune disease or syndrome. Alternatively, APC cell lines or clones that are able to appropriately process and/or present autoantigen to responder cells are preferred stimulator cells. In a particularly preferred embodiment, responder and stimulator cells are obtained from a source with diabetes or multiple sclerosis.

At this point, the responder T cells can be selectively amplified and/or stimulated, thereby producing a subset of T cells that are specific for the antigenic peptide. For instance, antigenic peptide-reactive responder cells may be selected by flow cytometry, and particularly by fluorescence activated cell sorting. This subset of responder cells can be maintained by repetitive stimulation with APCs presenting the same antigenic peptide. Alternatively, responder cell clones or lines can be established from this responder cell subset. Further, this subset of responder cells can be used to map epitopes of the antigenic peptide and the protein from which it is derived.

Other methods to assess the biological activity of the single chain MHC class II molecule:peptide complexes are known in the art and can be used herein, such as using a microphysiometer, to measure production of acidic metabolites in T cells following interaction with antigenic peptide. Other assay methods include competition assays, comparing single chain MHC class II molecule:complex response with that to the normal antigen. Also measurement production of such indicators as cytokines or γ interferon can provide an indication of complex response.

### Animal Models of Autoimmune Disease

Similar assays and methods can be developed for and used in animal models. For instance, the therapeutic effect of a pharmaceutical composition of the single chain molecule or multimer or a polynucleotide encoding the single chain molecule or multimer can be tested in vivo in a number of animal models of HLA-DR-associated autoimmune disease. These diseases include, but are not limited to, insulin-dependent diabetes mellitus, multiple sclerosis, myasthenia gravis, pernicious anemia, rheumatoid arthritis, and systemic lupus erythematosus.

For example, NOD mice are a spontaneous model of IDDM. Treatment with the pharmaceutical compositions prior to or after onset of disease can be monitored by assay of urine glucose levels in the NOD mouse, as well as by in vitro T cell proliferation assays to assess reactivity to known autoantigens (see, e.g., Kaufman et al., Nature 366:69-72 (1993)) for example). Alternatively, induced models of autoimmune disease, such as EAE, can be treated with pharmaceutical composition. Treatment in a preventive or intervention mode can be followed by monitoring the clinical symptoms of EAE.

Following is a description of several other animal models of HLA-DR-associated autoimmune disease which can be used to assay in vivo effects of the peptide. It will be obvious to one of skill in the art that other suitable animal models for autoimmune diseases can be utilized in a similar manner.

### Systemic Lupus Erythematosus (SLE)

F₁ hybrids of autoimmune New Zealand black (NZB) mice and the phenotypically normal New Zealand White (NZW) mouse strain develop severe systemic autoimmune disease, more fulminant than that found in the parental NZB strain. These mice manifest several immune abnormalities, including antibodies to nuclear antigens and subsequent development of a fatal, immune complex-mediated glomerulonephritis with female predominance, remarkably similar to SLE in humans (Knight et al., J. Exp. Med. 147:1653 (1978)), which is incorporated hereby by reference.

In both the human and murine forms of the disease, a strong association with MHC gene products has been reported. HLA-DR2 and HLA-DR3 individuals are at a higher risk than the general population to develop SLE (Reinertsen et al., N. Engl. J. Med. 299:515 (1970)), while in NZB/W F₁ mice (H-2^{d/u}), a gene linked to the h-2^{u} haplotype derived from the NZW parent contributes to the development of the lupus-like nephritis.

The effect of the invention can be measured by survival rates and by the progress of development of the symptoms, such as protenuria and appearance of anti-DNA antibodies.

Proteinuria can be measured by any method known to those of skill in the art, e.g. colorimetrically by the use of Uristix (Miles Laboratories, Inc., Elkhart, Ind.), giving an approximation of proteinuria as follows: trace, 10 mg/dl; 1+, 30 mg/dl; 100 mg/dl; 3+, 300 mg/dl; and 4+, 1000 mg/dl.

The presence of anti-DNA specific antibodies in NZB/W F₁ mice can be determined by using a modification of a linked immunosorbent assay (ELISA) described by Zouali et al., J. Immunol. Methods 90:105 (1986)) which is incorporated herein by reference.

### Myasthenia Gravis (MG)

Myasthenia gravis is one of several human autoimmune diseases linked to HLA-D (Safenberg, et al., Tissue Antigens 12:136 (1978); McDevitt et al, Arth. Rheum. 20:59 (1977)) which are incorporated herein by reference. In MG antibodies to the acetyl choline receptors (AcChoR) impair neuromuscular transmission by mediating loss of AcChoR in the postsynaptic membrane.

SJL/J female mice are a model system for human MG. In these animals, experimental autoimmune myasthenia gravis (EAMG) can be induced by immunizing the mice with soluble AcChoR protein from another species. Susceptibility to EAMG is linked in part to the MHC and has been mapped to the region within H-2 (Christadoss et al., J. Immunol. 123:2540 (1979)).

AcChoR protein can purified from Torpedo californica and assayed according to the method of Waldor et al., Proc. Natl. Acad. Sci. USA 80:2713 (1983), incorporated by reference. For example, emulsified AcChoR, 15 μg in complete Freund adjuvant, is injected intradermally among six sites on the back, the hind foot pads, and the base of the tail. Animals are reimmunized with this same regimen 4 weeks later.

Evaluation can be made by measurement of anti-AcChoR antibodies by any method known to those of skill in the art, e.g., a microtiter ELISA assay as described in Waldor et al., supra. In an exemplary assay, the standard reagent volume is 50 μper well. Reagents are usually incubated in the wells for 2 hr at RT. Five μg of AcChoR diluted in bicarbonate buffer, pH 9.6, is added to each well. After incubation with AcChoR, the plates are rinsed four times with a wash solution consisting of phosphate-buffer saline containing 0.05% Tween and 0.05% NaN₃. Mouse sera are diluted in 0.01M PBS (pH 7.2), 1.5 mfr MgCl₂, 2.0 mM 2-mercaptoethanol, 0.05% Tween-80, 0.05% NaN₃ (p-Tween buffer) and incubated on the plate. After the plate is washed, beta galactosidase-conjugated sheep anti-mouse antibody diluted in P-Tween buffer is added to each well. After a final washing, the enzyme substrate, p-nitrophenylgalctopyranoside is added to the plate, and the degree of substrate catalysis is determined from the absorbance at 405 nm after 1 hr.

Anti-AcChoR antibodies are expected to be present in the mice immunized with AcChoR as compared to nonimmunized mice. Treatment with complex is expected to significantly reduce the titer of anti-AcChoR antibodies in the immunized mice.

The effect of treatment with the invention on clinical EAMG can also be assessed by any method known to those of skill in the art. Myasthenia symptoms include a characteristic hunched posture with drooping of the head and neck, exaggerated arching of the back, splayed limbs, abnormal walking, and difficulty in righting. Mild symptoms are present after a standard stress test, and should be ameliorated by administration of complex.

### Rheumatoid Arthritis (RA)

In humans, susceptibility to rheumatoid arthritis is associated with HLA D/DR. The immune response in mice to native type II collagen has been used to establish an experimental model for arthritis with a number of histological and pathological features resembling human RA. Susceptibility to collagen-induced arthritis (CIA) in mice has been mapped to the H-2 I region, particularly the I-A subregion (Huse et al., Fed. Proc. 43:1820 (1984)).

Mice from a susceptible strain, DEA-1 can be caused to have CIA by treatment of the mice with native type II collagen, using the technique described in Wooley et al., J. Immunol. 134:2366 (1985), incorporated herein by reference.

In another model adjuvant arthritis in rats is an experimental model for human arthritis, and a prototype of autoimmune arthritis triggered by bacterial antigens (Holoschitz et al., Prospects of Immunology (1986); Pearson, Arthritis Rheum. 7:80 (1964)). The disease is the result of a cell-mediated immune response, as evidenced by its transmissibility by a clone of T cells which were reactive against the adjuvant (MT); the target self-antigen in the disease, based upon studies with the same cloned cells, appears to be part(s) of a proteoglycan molecule of cartilage.

Adjuvant disease in rats is produced as described by Pearson supra, i.e., by a single injection of Freund's adjuvant (killed tubercle bacilli or chemical fractions of it, mineral oil, and an emulsifying agent) given into several depot sites, preferably intracutaneously or into a paw or the base of the tail. The adjuvant is given in the absence of other antigens.

The effect of the invention treatment on manifestations of the disease can be monitored by any method known to those of skill in the art. These manifestations are histopathological, and include an acute and subacute synovitis with proliferation of synovial lining cells, predominantly a mononuclear infiltration of the articular and particular tissues, the invasion of bone and articular cartilage by connective tissue pannus, and periosteal new bone formation, especially adjacent to affected joints. In severe or chronic cases, destructive changes occur, as do fibrous or bony ankylosis. These histopathological symptoms are expected to appear in control animals at about 12 days after sensitization to the Freund's adjuvant.

### Insulin Dependent Diabetes Mellitus (IDDM)

IDDM is observed as a consequence of the selective destruction of insulin-secreting cells within the Islets of Langerhans of the pancreas. Involvement of the immune system in this disease is suggested by morphologic evidence of early infiltration of the Islets by mononuclear cells, by the detection of anti-islet cell antibodies, by the high frequency of HLA-DR3 and -DR4 alleles in IDDM populations, and by clinical associations between IDDM and various autoimmune diseases. An animal model for spontaneous IDDM and thyroiditis has been developed in the BB rat. As in humans, the rat disease is controlled in part by the genes encoding the MHC antigens, is characterized by islet infiltration, and is associated with the presence of anti-islet antibodies. The I-E equivalent class II MHC antigens appear to be involved in manifestation of the autoimmune diseases in the BB rat. Biotard et al., Proc. Natl. Acad. Sci. USA 82:6627 (1985).

In morphologic evaluation, insulitis is characterized by the presence of mononuclear inflammatory cells within the islets. Thyroiditis is characterized by focal interstitial lymphocytic infiltrate within the thyroid gland, as a minimum criterion. Most severe cases show diffuse extensive lymphocytic infiltrates, disruption of acini, fibrosis, and focal Hurthle call change. See Biotard et al. supra.

Treatment of the BB rats with the invention is expected to ameliorate or prevent the manifestation of the clinical and morphological symptoms associated with IDDM and thyroiditis.

In another model, the NOD mouse strain (H-2K^{d} D^{b}) is a murine model for autoimmune IDDM. The disease in these animals is characterized by anti-islet cell antibodies, severe insulitis, and evidence for autoimmune destruction of the beta-cells (Kanazawa, et al., Diabetolooia 27:113 (1984)). The disease can be passively transferred with lymphocytes and prevented by treatment with cyclosporin-A (Ikehara et al., Proc. Natl. Acad. Sci. USA 82:7743 (1985)); Mori et al., Diabetolooia 29:244 (1986). Untreated animals develop profound glucose intolerance and ketosis and succumb within weeks of the onset of the disease. Seventy to ninety percent of female and 20-30% of male animals develop diabetes within the first six months of life. Breeding studies have defined at least two genetic loci responsible for disease susceptibility, one of which maps to the MHC. Characterization of NOD Class II antigens at both the serologic and molecular level suggest that the susceptibility to autoimmune disease is linked to I-A_{B} (Acha-Orbea and McDevitt, Proc. Natl. Acad. Sci. USA 84:235 (1907)).

Treatment of Female NOD mice with complex is expected to lengthen the time before the onset of diabetes and/or to ameliorate or prevent the disease.

### Experimental Allergic Encephalomyelitis (EAE)

Experimental allergic encephalomyelitis (EAE) is an induced autoimmune disease of the central nervous system which is a model for multiple sclerosis (MS). The disease can be induced in many species, including mice and rats.

The disease is characterized by the acute onset of paralysis. Perivascular infiltration by mononuclear cells in the CNS is observed in both mice and rats. Methods of inducing the disease, as well as symptomology, are reviewed in Aranson, The Autoimmune Diseases (Rose and Mackay, eds., 1985), and in Acha-Orbea et al., Ann. Rev. Imm. 7:377-405 (1989).

One of the genes mediating susceptibility is localized in the MHC class II region (Moore et al., J. Immunol. 124:1815-1820 (1980)). The best analyzed encephalitogenic protein is myelin basic protein (MBP), but other encephalitogenic antigens are found in the brain. The immunogenic epitopes have been mapped (see, Acha-Orbea et al., supra.). In the PL mouse strains (H-2^{u}) two encephalitogenic peptides in MBP have been characterized: MBP peptide p35-47 (MBP 35-47), and acetylated NSF p1-9 (MBP 1-9).

The effect of the invention on ameliorating disease symptoms in individuals in which EAE has been induced can be measured by survival rates, and by the progress of the development of symptoms.

### Methods of Making the Complexes of the Invention

Expression systems suitable for production of appropriate recombinant single chain MHC class II molecule:peptide complexes are available and known in the art. Various prokaryotic, fungal, and eukaryotic host cells are suitable for expression of recombinatn, single chain MHC class II molecule:peptide complexes, as well as for individual recombinant alpha and beta MHC class II chains.

Prokaryotes that are useful as host cells, according to the present invention, most frequently are represented by various strains of Escherichia coli. However, other microbial strains can also be used, such as bacilli, for example Bacillus subtilis, various species of Pseudomonas, or other bacterial strains.

According to the invention, the single chain MHC class II molecule:peptide complexes are expressed from recombinantly engineered nucleotide sequences that encode the single chain MHC class II molecule:peptide polypeptides by operably linking the engineered nucleic acid coding sequence to signals that direct gene expression in prokaryotes. A nucleic acid is “operably linked” when it is placed into a functional relationship with another nucleic acid sequence. For instance, a promoter or enhancer is operably linked to a coding sequence if it effects the transcription of the sequence. Generally, operably linked means that the nucleic acid sequences being linked are contiguous and, where necessary to join two protein coding regions, contiguous and in reading frame.

The genes encoding the single chain MHC class II molecule:peptide complexes may be inserted into an “expression vector,” “cloning vector,” or “vector,” terms which are used interchangeably herein and usually refer to plasmids or other nucleic acid molecules that are able to replicate in a chosen host cell. Expression vectors may replicate autonomously, or they can replicate by being inserted into the genome of the host cell, by methods well known in the art. Vectors that replicate autonomously will have an origin of replication or autonomous replicating sequence (ARS) that is functional in the chosen host cell(s).

Plasmid vectors that contain replication sites and control sequences derived from a species compatible with the chosen host are used. For example, E. coli is typically transformed using derivatives of pBR322, a plasmid derived from E. coli species by Bolivar et al., Gene 2:95-113, 1977. Often, it is desirable for a vector to be usable in more than one host cell, e.g., in E. coli for cloning and construction, and in a Bacillus cell for expression.

The expression vectors typically contain a transcription unit or expression cassette that contains all the elements required for the expression of the DNA encoding the MHC molecule in the host cells. A typical expression cassette contains a promoter operably linked to the DNA sequence encoding a single chain MHC class II molecule:peptide complex and a ribosome binding site. The promoter is preferably positioned about the same distance from the heterologous transcription start site as it is from the transcription start site in its natural setting. As is known in the art, however, some variation in this distance can be accommodated without loss of promoter function. In addition to a promoter sequence, the expression cassette can also contain a transcription termination region downstream of the structural gene to provide for efficient termination. The termination region may be obtained from the same gene as the promoter sequence or may be obtained from a different gene.

Commonly used prokaryotic control sequences which are defined herein to include promoters for transcription initiation, optionally with an operator, along with ribosome binding site sequences, include such commonly used promoters as the beta-lactamase (penicillinase) and lactose (lac) promoter systems (Change et al., Nature 198:1056, 1977) and the tryptophan (trp) promoter system (Goeddel et al., Nucleic Acids Res. 8:4057-74, 1980) and the lambda-derived P_{L} promoter and N-gene ribosome binding site (Shimatake et al., Nature 292:128-32, 1981). Any available promoter system that functions in prokaryotes can be used.

Either constitutive or regulated promoters can be used in the present invention. Regulated promoters can be advantageous because the host cells can be grown to high densities before expression of the single chain MHC class II molecule:peptide complexes is induced. High level expression of heterologous proteins slows cell growth in some situations. Regulated promoters especially suitable for use in E. coli include the bacteriophage lambda P_{L} promoter, the hybrid trp-lac promoter (Amann et al., Gene 25:167-78 1983; and the bacteriophage T7 promoter.

For expression of single chain MHC class II molecule:peptide complexes in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

A ribosome binding site (RBS) is also necessary for expression of single chain MHC class II molecule:peptide complexes in prokaryotes. An RBS in E. coli, for example, consists of a nucleotide sequence 3-9 nucleotides in length located 3-11 nucleotides upstream of the initiation codon (Shine and Dalgamo, Nature , 254:34-40, 1975; Steitz, In Biological regulation and development: Gene expression (ed., Goldberger), vol. 1, p. 349, 1979).

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al., J. Biol. Chem. 263:16297-16302, 1988.

The single chain MHC class II molecule:peptide complexes can be expressed intracellularly, or can be secreted from the cell. Intracellular expression often results in high yields. However, some of the protein may be in the form of insoluble inclusion bodies. Although some of the intracellularly produced MHC polypeptides of the present invention may be active upon being harvested following cell lysis, the amount of soluble, active MHC polypeptide may be increased by performing refolding procedures using methods known to those of skill in the art (see, e.g., Sambrook et al., Molecular Cloning: A Laboratory Manual Second Edition, Cold Spring Harbor, N.Y., 1989.; Marston et al., Bio/Technology 2:800-804, 1985; Schoner et al., Bio/Technology 3:151-54, 1985). In one embodiment, for purification and refolding the cell pellet is lysed and refolded in urea-borate-DTT buffer followed by urea-borate buffer and reverse phase HPLC purification using either silica gel based Vydac (Hewlett Packard, Wilmington, Del.) or polymer based Poros-R2 (PerSeptive Biosystems) resins, with bead size varying based on the scale of the culture and is described in further detail below. In one embodiment, e.g., for large scale refolding, the sample can be ultrafiltered into a urea-borate buffer to which is then added 0.2 μM to 1 mM copper sulfate, preferably 0.2 to 20 μM, after which folding occurs immediately.

More than one MHC:peptide complex may be expressed in a single prokaryotic cell by placing multiple transcriptional cassettes in a single expression vector, or by utilizing different selectable markers for each of the expression vectors which are employed in the cloning strategy.

A second approach for expressing the MHC:peptide complexes of the invention is to cause the polypeptides to be secreted from the cell, either into the periplasm or into the extracellular medium. The DNA sequence encoding the MHC polypeptide is linked to a cleavable signal peptide sequence. The signal sequence directs translocation of the MHC:peptide complex through the cell membrane. An example of a suitable vector for use in E. coli that contains a promoter-signal sequence unit is pTA1S29, which has the E. coli phoA promoter and signal sequence see, e.g., Sambrook et al., supra; Oka et al., Proc. Natl. Acad. Sci. USA 82:7212-16, 1985; Talmadge et al., Proc. Natl. Acad. Sci. USA 77:39892, 1980; Takahara et al., J. Biol. Chem. 260: 2670-74, 1985). Once again, multiple polypeptides can be expressed in a single cell for periplasmic association. Eukaryotic signal sequences are also well known to those of skill in the art, and cause the MHC:peptide complexes of the invention to be secreted into the extracellular medium.

The MHC:peptide complexes of the invention can also be produced as fusion proteins. This approach often results in high yields, because normal prokaryotic control sequences direct transcription and translation. In E. coli, lacZ fusions are often used to express heterologous proteins. Suitable vectors are readily available, such as the pUR, pEX, and pMR100 series (see, e.g., Sambrook et al., supra). For certain applications, it may be desirable to cleave the non-MHC amino acids from the fusion protein after purification. This can be accomplished by any of several methods known in the art, including cleavage by cyanogen bromide, a protease, or by Factor X, (see, e.g., Sambrook et al., supra.; Goeddel et al., Proc. Natl. Acad. Sci. USA 76:106-10, 1979; Nagai et al., Nature 309:810-12, 1984; Sung et al., Proc. Natl. Acad. Sci. USA 83:561-65, 1986). Cleavage sites can be engineered into the gene for the fusion protein at the desired point of cleavage.

Foreign genes, such as single chain MHC class II molecule:peptide complexes, can be expressed in E. coli as fusions with binding partners, such as glutathione-S-transferase (GST), maltose binding protein, or thioredoxin. These binding partners are highly translated and can be used to overcome inefficient initiation of translation of eukaryotic messages in E. coli. Fusion to such binding partner can result in high-level expression, and the binding partner is easily purified and then excised from the protein of interest. Such expression systems are available from numerous sources, such as Invitrogen Inc. (San Diego, Calif.) and Pharmacia LKB Biotechnology Inc. (Piscataway, N.J.).

A method for obtaining recombinant proteins from E. coli which maintains the integrity of their N-termini has been described by Miller et al. Biotechnology 7:698-704 (1989). In this system, the gene of interest is produced as a C-terminal fusion to the first 76 residues of the yeast ubiquitin gene containing a peptidase cleavage site. Cleavage at the junction of the two moieties results in production of a protein having an intact authentic N-terminal reside.

The vectors containing the nucleic acids that code for the single chain MHC class II molecule:peptide complexes are transformed into prokaryotic host cells for expression. “Transformation” refers to the introduction of vectors containing the nucleic acids of interest directly into host cells by well known methods. The particular procedure used to introduce the genetic material into the host cell for expression of the single chain MHC class II molecule:peptide complex is not particularly critical. Any of the well known procedures for introducing foreign nucleotide sequences into host cells may be used. It is only necessary that the, particular host cell utilized be capable of expressing the gene.

Transformation methods, which vary depending on the type of the prokaryotic host cell, include electroporation; transfection employing calcium chloride, rubidium chloride calcium phosphate, or other substances; microprojectile bombardment; infection (where the vector is an infectious agent); and other methods. See, generally, Sambrook et al, supra, and Ausubel et al., (eds.) Current Protocols in Molecular Biology, John Wiley and Sons, Inc., NY, 1987. Reference to cells into which the nucleic acids described above have been introduced is meant to also include the progeny of such cells. Transformed prokaryotic cells that contain expression vectors for single chain MHC class II molecule:peptide complexes are also included in the invention.

After standard transfection or transformation methods are used to produce prokaryotic cell lines that express large quantities of the single chain MHC class II molecule:peptide complex polypeptide, the polypeptide is then purified using standard techniques. See, e.g., Colley et al., J. Chem. 64:17619-22, 1989; and Methods in Enzymology, “Guide to Protein Purification”, Deutscher, ed., Vol. 182 (1990). The recombinant cells are grown and the single chain MHC class II molecule:peptide complex is expressed. The purification protocol will depend upon whether single chain MHC class II molecule:peptide complex is expressed intracellularly, into the periplasm, or secreted from the cell. For intracellular expression, the cells are harvested, lysed, and the polypeptide is recovered from the cell lysate (Sambrook et al., supra). Periplasmic MHC polypeptide is released from the periplasm by standard techniques (Sambrook et al., supra). If the MHC polypeptide is secreted from the cells, the culture medium is harvested for purification of the secreted protein. The medium is typically clarified by centrifugation or filtration to remove cells and cell debris.

The MHC polypeptides can be concentrated by adsorption to any suitable resin (such as, for example, CDP-Sepharose, Asialoprothrombin-Sepharose 4B, or Q Sepharose), or by use of ammonium sulfate fractionation, polyethylene glycol precipitation, or by ultrafiltration. Other means known in the art may be equally suitable.

Further purification of the MHC polypeptides can be accomplished by standard techniques, for example, affinity chromatography, ion exchange chromatography, sizing chromatography, reverse phase HPLC, or other protein purification techniques used to obtain homogeneity. The purified proteins are then used to produce pharmaceutical compositions.

For secretion of a polypeptide or protein of interest, recombinant nucleic acid constructs of the invention may include sequences that encode signal sequences or other sequences that direct secretion. Secretory signal sequences, also called leader sequences, prepro sequences and/or pre sequences, are amino acid sequences that play a role in secretion of mature polypeptides or proteins from a cell. Such sequences are characterized by a core of hydrophobic amino acids and are typically (but not exclusively) found at the amino termini of newly synthesized proteins. The secretory signal sequence may be that of the protein of interest, or may be derived from another secreted protein (e.g., t-PA, a preferred mammalian secretory leader) or synthesized de novo. The secretory signal sequence is joined to the DNA sequence encoding a protein of the present invention in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830). Very often the secretory peptide is cleaved from the mature protein during secretion. Such secretory peptides contain processing sites that allow cleavage of the secretory peptide from the mature protein as it passes through the secretory pathway. An example of such a processing site is a dibasic cleavage site, such as that recognized by the Saccharomyces cerevisiae KEX2 gene or a Lys-Arg processing site. Processing sites may be encoded within the secretory peptide or may be added to the peptide by, for example, in vitro mutagenesis.

Secretory signals include the α factor signal sequence (prepro sequence: Kurjan & Herskowitz, Cell 3:933-943, 1982; Kurjan et al., U.S. Pat. No. 4,546,082; Brake, EP 116, 201), the PHO5 signal sequence (Beck et al., WO 86/00637), the BAR1 secretory signal sequence (MacKay et al., U.S. Pat. No. 4,613,572; MacKay, WO 87/002670), the SUC2 signal sequence (Carlsen et al., Molecular and Cellular Biology 3: 439-447, 1983), the a-1-antitrypsin signal sequence (Kurachi et al., Proc. Natl. Acad. Sci. USA 78: 6826-6830, 1981), the a-2 plasmin inhibitor signal sequence (Tone et al., J. Biochem. (Tokyo) 102: 1033-1042, 1987) and the tissue plasminogen activator signal sequence (Pennica et al., Nature 301: 214-221, 1983). Alternately, a secretory signal sequence may be synthesized according to the rules established, for example, by von Heinje (European Journal of Biochemistry 133: 17-21, 1983; Journal of Molecular Biology 184: 99-105, 1985; Nucleic Acids Research 14: 4683-4690; 1986). Another signal sequence is the synthetic signal LaC212 spx (1-47). ERLE described in WO 90/10075.

Secretory signal sequences may be used singly or may be combined. For example, a first secretory signal sequence may be used in combination with a sequence encoding the third domain of barrier (described in U.S. Pat. No. 5,037,243, which is incorporated by reference herein in its entirety). The third domain of barrier may be positioned in proper reading frame 3′ of the DNA segment of interest or 5′ to the DNA segment and in proper reading frame with both the secretory signal sequence and a DNA segment of interest.

The choice of suitable promoters, terminators and secretory signals for all expression systems, is well within the level of ordinary skill in the art. Methods for expressing cloned genes in Saccharomyces cerevisiae are generally known in the art (see, “Gene Expression Technology,” Methods in Enzymology, Vol. 185, Goeddel (ed.), Academic Press, San Diego, Calif., 1990 and “Guide to Yeast Genetics and Molecular Biology, “Methods in Enzymology, Guthrie and Fink (eds.), Academic Press, San Diego, Calif., 1991; which are incorporated herein by reference). Proteins of the present invention can also be expressed in filamentous fungi, for example, strains of the fungi Aspergillus (McKnight et al., U.S. Pat. No. 4,935,349, which is incorporated herein by reference). Expression of cloned genes in cultured mammalian cells and in E. coli, for example, is discussed in detail in Sambrook et al. (Molecular Cloning: A Laboratory Manual. Second Edition, Cold Spring Harbor, N.Y., 1989; which is incorporated herein by reference). As would be evident to one skilled in the art, one could express the proteins of the instant invention in other host cells such as avian, insect and plant cells using regulatory sequences, vectors and methods well established in the literature.

In yeast, suitable yeast vectors for use in the present invention include YRp7 (Struhl et al., Proc. Natl. Acad. Sci. USA 76:1035-1039, 1978), YEpl3 (Broach et al., Gene 8: 121-133, 1979), POT vectors (Kawasaki et al., U.S. Pat. No. 4,931,373, which is incorporated by reference herein), pJDB249 and pJDB219 (Beggs, Nature 275:104-108, 1978) and derivatives thereof. Preferred promoters for use in yeast include promoters from yeast glycolytic genes (Hitzeman et al., J. Biol. Chem. 255: 12073-12080, 1980; Alber and Kawasaki, J. Mol. Appl. Genet. 1: 419-434, 1982; Kawasaki, U.S. Pat. No. 4,599,311) or alcohol dehydrogenase genes (Young et al., in Genetic Engineering of Microorganisms for Chemicals, Hollaender et al., (eds.), p. 355, 1982; Ammerer, Meth. Enzymol. 101: 192-201, 1983). Other promoters are the TPI1 promoter (Kawasaki, U.S. Pat. No. 4,599,311, 1986) and the ADH2-4^{c} promoter (Russell et al., Nature 304: 652-654, 1983; Irani and Kilgore, U.S. patent application Ser. No. 07/784,653, CA 1,304,020 and EP 284 044, which are incorporated herein by reference). The expression units may also include a transcriptional terminator such as the TPI1 terminator (Alber and Kawasaki, ibid.).

Yeast cells, particularly cells of the genus Pichia or Saccharomyces, are a preferred host for use in producing compound of the current invention. Methods for transforming yeast cells with exogenous DNA and producing recombinant proteins therefrom are disclosed by, for example, Kawasaki, U.S. Pat. No. 4,599,31 1; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075, which are incorporated herein by reference. Transformed cells are selected by phenotype determined by a selectable marker, commonly drug resistance or the ability to grow in the absence of a particular nutrient (e.g., leucine). A preferred vector system for use in yeast is the POT1 vector system disclosed by Kawasaki et al. (U.S. Pat. No. 4,931,373), which allows transformed cells to be selected by growth in glucose-containing media. A preferred secretory signal sequence for use in yeast is that of the S. cerevisiae MFα1 gene (Brake, ibid.; Kurjan et al., U.S. Pat. No. 4,546,082). Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092, which are incorporated herein by reference) and alcohol dehydrogenase genes. See also U.S. Pat. Nos. 4,990,446; 5,063,154; 5,139,936 and 4,661,454, which are incorporated herein by reference. Transformation systems for other yeasts, including Hansenula polymorpha, Schizosaccharomyces pombe, Kluyveromyces lactis, Kluyveromycesfragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986; Cregg, U.S. Pat. No. 4,882,279; and Stroman et al., U.S. Pat. No. 4,879,231.

Other fungal cells are also suitable as host cells. For example, Aspergillus cells may be utilized according to the methods of McKnight et al., U.S. Pat. No. 4,935,349, which is incorporated herein by reference. Methods for transforming Acremonium chrysogenum are disclosed by Sumino et al., U.S. Pat. No. 5,162,228, which is incorporated herein by reference. Methods for transforming Neurospora are disclosed by Lambowitz, U.S. Pat. No. 4,486,533, which is incorporated herein by reference.

Host cells containing DNA constructs of the present invention are then cultured to produce the heterologous proteins. The cells are cultured according to standard methods in a culture medium containing nutrients required for growth of the particular host cells. A variety of suitable media are known in the art and generally include a carbon source, a nitrogen source, essential amino acids, vitamins, minerals and growth factors. The growth medium will generally select for cells containing the DNA construct by, for example, drug selection or deficiency in an essential nutrient which is complemented by a selectable marker on the DNA construct or co-transfected with the DNA construct.

Yeast cells, for example, are preferably cultured in a chemically defined medium, comprising a non-amino acid nitrogen source, inorganic salts, vitamins and essential amino acid supplements. The pH of the medium is preferably maintained at a pH greater than 2 and less than 8, preferably at pH 6.5. Methods for maintaining a stable pH include buffering and constant pH control, preferably through the addition of sodium hydroxide. Preferred buffering agents include succinic acid and Bis-Tris (Sigma Chemical Co., St. Louis, Mo.). Yeast cells having a defect in a gene required for asparagine-linked glycosylation are preferably grown in a medium containing an osmotic stabilizer. A preferred osmotic stabilizer is sorbitol supplemented into the medium at a concentration between 0.1 M and 1.5 M, preferably at 0.5 M or 1.0 M. Cultured mammalian cells are generally cultured in commercially available serum-containing or serum-free media. Selection of a medium appropriate for the particular host cell used is within the level of ordinary skill in the art.

Methods for introducing exogenous DNA into mammalian host cells include calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-45, 1982) and DEAE-dextran mediated ‘transfection (Ausubel et al., (eds), Current Protocols in Molecular Biology, John Wiley and Sons, Inc., NY, 1987), which are incorporated herein by reference. Cationic lipid transfection using commercially available reagents, including the Boehringer Mannheim TRANSFECTION-REAGENT (N-[1-(2,3-dioleoyloxy)propyl]-N,N,N-trimethyl ammoniummethylsulfate; Boehringer Mannheim, Indianapolis, Ind.) or LIPOFECTIN reagent (N-[1-(2,3-dioleoyloxy)propyl]-N,N,N-trimethylammonium chloride and dioleoyl phosphatidylethanolamine; GIBCO-BRL, Gaithersburg, Md.) using the manufacturer-supplied directions, may also be used. A preferred mammalian expression plasmid is Zem229R (deposited under the terms of the Budapest Treaty with American Type Culture Collection, 12301 Parklawn Drive, Rockville, Md. on Sep. 28, 1993 as an E. coli HBO101transformant and assigned Accession Number 69447). The production of recombinant proteins in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134, which are incorporated herein by reference. Preferred cultured mammalian cells include the COS-1 (ATCC No. CRL 1650), COS-7 (ATCC No. CRL 1651), BHK (ATCC No. CRL 1632), BHK 570 (ATCC No. CRL 10314), DG44, and 293 (ATCC No. CRL 1573; Graham et al., J. Gen. Virol. 36:59-72, 1977) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978, which are incorporated herein by reference) and the adenovirus major late promoter.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used.

The soluble, fused MHC:peptide complexes of the present invention can be purified by first isolating the polypeptides from the cells followed by conventional purification methods, such as by ion-exchange and partition chromatography as described by, for example, Coy et al. (Peptides Structure and Function, Pierce Chemical Company, Rockford, Ill., pp 369-72, 1983) or by reverse-phase chromatography as described, for example, by Andreu and Merrifield (Eur. J. Biochem. 164: 585-90, 1987), or by HPLC as described, for example, by Kofod et al. (Int. J. Peptide and Protein Res. 32.: 436-40, 1988). Additional purification can be achieved by additional conventional purification means, such as liquid chromatography, gradient centrifugation, and gel electrophoresis, among others. Methods of protein purification are known in the art (see generally, Scopes, R., Protein Purification, Springer-Verlag, NY, 1982, which is incorporated by reference herein) and can be applied to the purification of the recombinant polypeptides described herein. Single chain MHC class II molecule:peptide complexes of at least about 50% purity are preferred, at least about 70-80% purity more preferred, and about 95-99% or more purity most preferred, particularly for pharmaceutical uses. Once purified, either partially or to homogeneity, as desired, the single chain MHC class II molecule:peptide complexes may then be used diagnostically or therapeutically, as further described below.

### Methods of Using Single Chain MHC Class II Molecule:peptide Complexes

The single chain MHC class II molecule:peptide complexes of the present invention may be used within methods for down-regulating parts of the immune system that are reactive in autoimmune diseases. The single chain MHC class II molecule:peptide complexes of the present invention are contemplated to be advantageous for use as immunotherapeutics to induce immunological tolerance or nonresponsiveness (anergy) in patients predisposed to mount or already mounting an immune response those particular autoantigens. A patient having or predisposed to a particular autoimmune disease is identified and MHC type is determined by methods known in the art. The patient's T cells can be examined in vitro to determine autoantigenic peptide(s) recognized by the patient's autoreactive T cells using complexes and methods described herein. The patient can then be treated with complexes of the invention. Such methods will generally include administering single chain MHC class II molecule:peptide complex in an amount sufficient to lengthen the time period before onset of the autoimmune disease and/or to ameliorate or prevent that disease. Single chain MHC class II molecule:peptide complexes of the present invention are therefore contemplated to be advantageous for use in both therapeutic and diagnostic applications related to auto immune diseases.

Kits can also be supplied for therapeutic or diagnostic uses. Thus, the subject composition of the present invention may be provided, usually in a lyophilized form, in a container. The single chain MHC class II molecule:peptide complex is included in the kits with instructions for use, and optionally with buffers, stabilizers, biocides, and inert proteins. Generally, these optional materials will be present at less than about 5% by weight, based on the amount of single chain MHC class II molecule:peptide complex, and will usually be present in a total amount of at least about 0.001% by weight, based on the single chain MHC class II molecule:peptide complex concentration. It may be desirable to include an inert extender or excipient to dilute the active ingredients, where the excipient may be present in from about 1 to 99% weight of the total composition.

Within one aspect of the present invention, single chain MHC class II molecule:peptide complexes are utilized to prepare antibodies for diagnostic or therapeutic uses. As used herein, the term “antibodies” includes polyclonal antibodies, monoclonal antibodies, antigen-binding fragments thereof such as F(ab′)₂ and Fab fragments, as well as recombinantly produced binding partners. These binding partners incorporate the variable or CDR regions from a gene which encodes a specifically binding antibody. The affinity of a monoclonal antibody or binding partner may be readily determined by one of ordinary skill in the art (see, Scatchard, Ann. NY Acad. Sci. 51: 660-72, 1949)

Methods for preparing polyclonal and monoclonal antibodies have been well described in the literature (see, for example, Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Ind., Boca Raton, Fla., 1982, which is incorporated herein by reference) . As would be evident to one of ordinary skill in the art, polyclonal antibodies may be generated from a variety of warm-blooded animals, such as horses, cows, goats, sheep, dogs, chickens, rabbits, mice, or rats, for example. The immunogenicity of the single chain MHC class II molecule:peptide complexes may be increased through the use of an adjuvant, such as Freund's complete or incomplete adjuvant. A variety of assays known to those skilled in the art may be utilized to detect antibodies which specifically bind to a single chain MHC class II molecule:peptide complex. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radio-immunoassays, radio-immunoprecipitations, enzyme-linked immuno-sorbent assays, dot blot assays, inhibition or competition assays, and sandwich assays.

Additional techniques for the preparation of monoclonal antibodies may be utilized to construct and express recombinant monoclonal antibodies. Briefly, mRNA is isolated from a B cell population and used to create heavy and light chain immunoglobulin cDNA expression libraries in a suitable vector such as the λIMMUNOZAP(H) and λIMMUNOZAP(L) vectors, which may be obtained from Stratagene Cloning Systems (La Jolla, Calif.). These vectors are then screened individually or are co-expressed to form Fab fragments or antibodies (Huse et al., Science 246 1275-81, 1989; Sastry et al., Proc. Natl. Acad. Sci. USA 86: 5728-32, 1989). Positive plaques are subsequently converted to a non-lytic plasmid which allows high level expression of monoclonal antibody fragments in E. coli.

Antibodies of the present invention may be produced by immunizing an animal selected from a wide variety of warm-blooded animals, such as horses, cows, goats, sheep, dogs, chickens, rabbits, mice, and rats, with a recombinant single chain MHC class II molecule:peptide complex. Serum from such animals are a source of polyclonal antibodies. Alternatively antibody producing cells obtained from the immunized animals are immortalized and screened. As the generation of human monoclonal antibodies to a human antigen, such as a single chain MHC class II molecule:peptide complex, may be difficult with conventional immortalization techniques, it may be desirable to first make non-human antibodies. Using recombinant DNA techniques, the antigen binding regions of the non-human antibody is transferred to the corresponding site of a human antibody coding region to produce a substantially human antibody molecules. Such methods are generally known in the art and are described in, for example, U.S. Pat. No. 4,816,397, and EP publications 173,494 and 239,400, which are incorporated herein by reference.

In another aspect of the invention, the single chain MHC class II molecule:peptide complexes can be used to clone T cells which have specific receptors for the single chain MHC class II molecule:peptide complex. Once the single chain MHC class II molecule:peptide complex-specific T cells are isolated and cloned using techniques generally available to the skilled artisan, the T cells or membrane preparations thereof can be used to immunize animals to produce antibodies to the single chain MHC class II molecule:peptide complex receptors on T cells. The antibodies can be polyclonal or monoclonal. If polyclonal, the antibodies can be murine, lagomorph, equine, ovine, or from a variety of other mammals. Monoclonal antibodies will typically be murine in origin, produced according to known techniques, or human, as described above, or combinations thereof, as in chimeric or humanized antibodies. The anti-single chain MHC class II molecule:peptide complex receptor antibodies thus obtained can then be administered to patients to reduce or eliminate T cell subpopulations that display such receptor. This T-cell population recognizes and participates in the immunological destruction of cells bearing the autoantigenic peptide in an individual predisposed to or already suffering from a disease, such as an autoimmune disease related to the autoantigenic peptide.

The coupling of antibodies to solid supports and their use in purification of proteins is well known in the literature (see, for example, Methods in Molecular Biology. Vol. 1, Walker (Ed.), Humana Press, New Jersey, 1984, which is incorporated by reference herein in its entirety). Antibodies of the present invention may be used as a marker reagent to detect the presence of MHC heterodimer:peptide complexes on cells or in solution. Such antibodies are also useful for Western analysis or immunoblotting, particularly of purified cell-secreted material. Polyclonal, affinity purified polyclonal, monoclonal and single chain antibodies are suitable for use in this regard. In addition, proteolytic and recombinant fragments and epitope binding domains can be used herein. Chimeric, humanized, veneered, CDR-replaced, reshaped or other recombinant whole or partial antibodies are also suitable.

### Pharmaceutical Compositions

Pharmaceutically acceptable carriers are determined in part by the particular composition being administered (e.g., nucleic acid, protein) as by the particular method used to administer the composition. Accordingly, there are a wide variety of suitable formulations of pharmaceutical compositions of the present invention (see, e.g., Remington's Pharmaceutical Sciences (17^{th} ed., 1989). Administration can be in any convenient manner, e.g., by injection, oral administration, inhalation, or transdermal application.

Formulations suitable for oral administration can consist of (a) liquid solutions, such as an effective amount of the packaged nucleic acid or polypeptide suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The polypeptides of the invention are administered prophylactically or to an individual already suffering from the disease. The compositions are administered to a patient in an amount sufficient to elicit an effective immune response. An amount adequate to accomplish this is defined as “therapeutically effective dose” or “immunogenically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization dose (that is for therapeutic or prophylactic administration) from about 0.01 mg to about 50 mg per 70 kilogram patient, more commonly from about 0.5-1 mg to about 10-15 mg per 70 kg of body weight. Boosting dosages are typically from about 0.01 mg to about 50 mg of peptide, more commonly about 0.5-1 mg to about 10-15 mg, using a boosting regimen over weeks to months depending upon the patient's response and condition. A suitable protocol would include injection at time 0, 2, 6, 8, 10 and 14 weeks, followed by booster injections at 24 and 28 weeks. Booster injections can be from one, two, three, four, five or more. Initial and booster injection amounts and timing are determined based on the judgment of the physician and the antigen being administered. In one embodiment, the initial and booster dose is 1.3 mg, 4 mg, or 13 mg, administered via intramuscular injection, with at least one and up to 3 booster injections at 8 week intervals, or at least one and up to 4 booster injections at 6 week intervals.

The therapeutic methods of the present invention may involve oral tolerance (Weiner et al., Nature 376: 177-80, 1995), or intravenous tolerance, for example. Tolerance can be induced in mammals, although conditions for inducing such tolerance will vary according to a variety of factors. To induce immunological tolerance in an adult susceptible to or already suffering from an autoantigen-related disease such as IDDM, the precise amounts and frequency of administration will also vary. For instance for adults about 20-80 μg/kg can be administered by a variety of routes, such as parenterally, orally, by aerosols, intradermal injection, and the like. For neonates, tolerance can be induced by parenteral injection or more conveniently by oral administration in an appropriate formulation. The precise amount administrated, and the mode and frequency of dosages, will vary.

The single chain MHC class II molecule:peptide complexes will typically be more tolerogenic when administered in a soluble form, rather than in an aggregated or particulate form. Persistence of a single chain MHC class II molecule:peptide complex of the invention is generally needed to maintain tolerance in an adult, and thus may require more frequent administration of the complex, or its administration in a form which extends the half-life of the complex. See for example, Sun et al., Proc. Natl. Acad. Sci. USA 91: 10795-99, 1994.

Within another aspect of the invention, a pharmaceutical composition is provided which comprises a single chain MHC class II molecule:peptide complex of the present invention contained in a pharmaceutically acceptable carrier or vehicle for parenteral, topical, oral, or local administration, such as by aerosol or transdermally, for prophylactic and/or therapeutic treatment, according to conventional methods. The composition may typically be in a form suited for systemic injection or infusion and may, as such, be formulated with sterile water or an isotonic saline or glucose solution. Formulations may further include one or more diluents, fillers, emulsifiers, preservatives, buffers, excipients, and the like, and may be provided in such forms as liquids, powders, emulsions, suppositories, liposomes, transdermal patches and tablets, for example.

Pharmaceutical compositions of the present invention are administered at daily to weekly intervals. An “effective amount” of such a pharmaceutical composition is an amount that provides a clinically significant decrease in a deleterious T cell-mediated immune response to an autoantigen, for example, those associated with IDDM, or provides other pharmacologically beneficial effects. Such amounts will depend, in part, on the particular condition to be treated, age, weight, and general health of the patient, and other factors evident to those skilled in the art. Preferably the amount of the single chain MHC class II molecule:peptide complex administered will be within the range of 20-80 μg/kg. Compounds having significantly enhanced half-lives may be administered at lower doses or less frequently.

### Adjuvants

An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

The compositions may also include a Mycobacterium species CWS adjuvant, as described above. The effectiveness of an adjuvant may be determined by measuring the amount of antibodies directed against the immunogenic peptide.

Certain adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins . Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol® to increase viscosity, or may be formulated in a dry powder form with a powder excipient such as lactose.

In one embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other formulations comprise an oil-in-water emulsion and tocopherol. Another adjuvant formulation employs QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 as disclosed in WO 00/09159. Preferably the formulation additionally comprises an oil in water emulsion and tocopherol.

Additional illustrative adjuvants for use in the pharmaceutical compositions of the invention include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula (I):

HO(CH₂CH₂O)ₙ-A-R,

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

### Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into the subjects. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the compositions disclosed herein. The formation and use of liposomes is generally known to those of skill in the art (see, for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516, specifically incorporated herein by reference in its entirety). Further, various methods of liposome and liposome-like preparations as potential drug carriers have been reviewed (Takakura, 1998; Chandran et al., 1997; Margalit, 1995; U.S. Pat. No. 5,567,434; U. S. Pat. No. 5,552,157; U. S. Pat. No. 5,565,213; U. S. Pat. No. 5,738,868 and U. S. Pat. No. 5,795,587, each specifically incorporated herein by reference in its entirety).

Liposomes have been used effectively to introduce genes, drugs (Heath & Martin, 1986; Heath et al., 1986; Balazsovits et al., 1989; Fresta & Puglisi, 1996), radiotherapeutic agents (Pikul et al., 1987), enzymes (Imaizumi et al., 1990a; Imaizumi et al., 1990b), viruses (Faller & Baltimore, 1984), transcription factors and allosteric effectors (Nicolau & Gersonde, 1979) into a variety of cultured cell lines and animals. In addition, several successful clinical trials examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori & Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the compositions. They are widely suitable as both water- and lipid-soluble substances can be entrapped, i.e., in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977, 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution. However, a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for hours or days, depending on their composition, and half lives in the blood range from minutes to several hours. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominant site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U. S. Pat. No. 5,145,684, specifically incorporated herein by reference in its entirety).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

The following example is provided by way of illustration only and not by way of limitation. Those of skill in the art will readily recognize a variety of noncritical parameters that could be changed or modified to yield essentially similar results.

### EXAMPLES

### Example 1

### Construction of DNA Sequences Encoding Human Single Chain MHC Class II :Peptide Complexes HLA-DR4 Molecules(CO563 and CO564).

DNA constructs encoding gp39 β1/α1 human molecules were prepared from a cDNA encoding the gp39 peptide fused to the β1/α1 domains of HLA-DR4 according to standard techniques. For the production of “empty” β1/α1 DR4 molecules, a cDNA encoding the fused β1/α1 domains of HLA-DR4 was prepared using cloned α and β chains from DR4.

Amino acid sequence of the gp39-β1/α1 HLA-DR4 human single chain molecule (linkers are shown in bold)

MGDTGRSFTLASSETGVGASGGGGSGGGGDTRPRFLEQVKHECH
FFNGTERVRFLDRYFYHQEEYVRFDSDVGEYRAVTELGRPDAEYWNSQKDLLEQ
KRAAVDTYCRHNYGVGESFTVQRRGGIKEEHVIIQAEFYLNPDQSGEFMFDFDG
DEIFHVDMAKKETVWRLEEFGRFASFEAQGALANIAVDKANLEIMTKRSNYTPIT
N*

N*

aa1-aa4: leader sequence

aa5-aa18: gp39 peptide

aa19-aa28: linker

aa29-aa122: HLA-DR4 β1 domain

aa123-aa124: linker

aa125-aa208: HLA-DR4 α1 domain

Amino acid sequence of the “empty” β1/α1 HLA-DR4 human single chain molecule (linkers in bold):

MGDTRPRFLEQVKHECHFFNGTERVRFLDRYFYHQEEYVRFDSDVGEYRAVTEL
GRPDAEYWNSQRDLLEQKRAAVDTYCRHNYGVGESFTVQRRGGIKEEHVIIQAE
FYLNPDQSGEFMFDFDGDEIFHVDMAKKETVWRLEEFGRFASFEAQGALANIAV
DKANLEIMTKRSNYTPITN*

aa 1-aa95: HLA-DR4 β1 domain

aa96-aa97: linker

aa98-aa181: HLA-DR4 α1 domain

For the production of recombinant proteins, the bacteria (pLysS) were grown in LB (containing ampicillin (50 μg/ml) and chloramphenicol (5 μg/ml)) at 37° C. until OD 600=0.5. IPTG was added at the final concentration of 0.5 mM final and the bacteria were further incubated for 3 hours at 37° C. with shaking. The bacteria were centrifuged at 4° C., 4000×g for 20 min and the pellet was frozen at −80° C. The following day, the pellet was resuspended in 40 ml of lysis buffer (50 mM Tris-HCl pH 8, 50 mM NaCl, 2 mM EDTA, 1 protease inhibitor cocktail tablet, 1% Triton X100 and 1% deoxycholate), and incubated for 1 hour at 4° C. under continuous agitation with a magnetic stirrer. The sample was then homogenized using a French Press with a 16,000 psi setting, and centrifuged at 4° C., 9000 g for 20 min. The pellet was then resuspended in 30 ml of lysis buffer without Triton and deoxycholate and centrifuged at 4° C., 9000 g for 20 min. The new pellet was resuspended in 10 ml of 20 mM ethanolamine/6 M urea pH 10, and eventually frozen at −80° C. The recombinant protein was then purified by FPLC ion-exchange chromatography using Source 30Q anion-exchange media in an XK26/20 column using a step gradient going from 1 mM to 1 M NaCl in 20 mM ethanolamine/6M urea pH 10. Fractions were analyzed by SDS/PAGE and those corresponding to the proteins of interest are pooled and dialyzed against PBS 1X.

### Example 2

### Production of Additional Single Chain Constructs

Additional constructs with different composition and length of the 2^{nd} linker (between β1 and α1) were engineered by using standard techniques using CO567 as the template. Specifically, PCR primers were designed to replace the old sequence in CO567 with the new sequence. For example, to make CO581, the primers were designed with the following sequences (note these primers were phosphorylated at 5′).

Primer 1:

5′pCACCAGGAGGAGAGCCGCCCACGCCGGTCTCGCTGG

Primer 2:

5′pGACCACCTGGATCTGGGGACACCCGACCACGTTTC

PCR reaction (100 μl) was made of the following components: 2 μl CO567 (80 ng) as template, 2 μl each of primer 1 and primer 2 (10 μM), 2 μl of dNTP mix (20 mM each), 10 μl of 10×pfu buffer, and 80 μl of sterile water. After all the components were mixed, 2 μl of Turbo pfu (5U total ) was added, mixed and put on PCR machine. The PCR cycles has a pre-denaturation at 95° C. for 30 sec, then 10 cycles of 95° C. for 30° C., 60° C. for 1 min, and 72° C. for 7 min. Then another 22 cycles 30° C., 65° C. for 1 min and 72° C. for 7 min, followed by a final 10 min at 72° C.

The PCR mixture was digested with 2 μl of DpnI (10U) for 2 h at 37° C. Then the PCR product at ˜6 kb was purified from agarose gel after electrophoresis. The purified PCR product was ligated by T4 DNA ligase for 1 h at room temperature then used to transform into NovaBlue (Novagen)competent cells by standard protocol. Cells were plated on LB (+Carb) and grow overnight at 37° C.

Next day, about a dozen single colony from the transformation were randomly picked for overnight culture in 5 ml LB (+Carb) at 37° C. Plasmids from these culture were purified with Wizard Miniprep kit, and analyzed by Xho I digestion. A few plasmids that passed the Xho I digestion were further confirmed by DNA sequencing.

To express the recombinant proteins, a clone with confirmed DNA sequence was used to transform BL21(DE3)CodonPlus-RIL (Stratagene) by standard transformation protocol and plated on LB (Carb+Cam) plates overnight at 37° C. Next morning, a single colony was picked to inoculate 100 ml LB (+Carb +Cam) and the culture was grown till OD reach between 0.8-1.0, and stored overnight at 4° C. Next day, the culture was pellet down and used to inoculate into 2×YT (+Carb+Cam) at ratio of 25 ml culture per liter new media. These large cultures were grown at 37° C. till OD=0.5-0.6, and IPTG was added to induce recombinant protein at 37° C. for 3 h. The induced cultures were pellet and stored at −80° C. till purification.

Table 1 provides a listing of various constructs made according to the invention.

| Construct | peptide | upstream linker | downstream linker |
|---|---|---|---|
| CO523 | yes | GGGG | GG |
| CO543 | none | none | GG |
| CO563 | yes | ASGGGSGGG | GG |
| CO567 | yes | ASGGGSGGG | TSGGGGSGGGGSSS |
| CO580 | yes | ASGGGSGGG | GSPGGGGSGGGPGS |
| CO581 | yes | ASGGGSGGG | GSPPGGPPGS |
| CO582 | yes | ASGGGSGGG | GSPGGGGPGS |
| CO583 | yes | ASGGGSGGG | TSGGGGS |
| CO584 | yes | ASGGGSGGG | SGGSGGS |
| CO585 | yes | ASGGGSGGG | FDAPSPLP |
| CO586 | none | none | TSGGGGSGGGGSSS |
| CO587 | none | none | GSPGGGGSGGGPGS |
| CO588 | none | none | GSPPGGPPGS |
| CO589 | none | none | GSPGGGGPGS |
| CO590 | none | none | TSGGGGS |
| CO591 | none | none | SGGSGGS |
| CO592 | none | none | FDAPSPLP |
| CO593 | yes | ASGGGSGGG | VYPEVTV |
| CO594 | none | none | VYPEVTV |
| CO595 | yes | ASGGGSGGG | GGGO |
| CO596 | yes | ASGGGSGGG | GGGGS |
| CO597 | yes | ASCGGSGGG | GGGSGG |

### Example 3

### Four Classes of Novel Linkers for MHC Class II Single Chain Molecules

Inspection of circular dichroism spectra of purified, refolded single chain constructs indicated that novel linkers could be used for the constructs. The atomic structures of the various murine and human MHC class II molecules, as determined by X-ray crystallography, indicated that these molecules have a high degree of structural similarity. The circular dichroism results were consistent with two folded molecules of clearly different secondary structure. Careful inspection of the structures reveals that the human MHC has a longer distance between chains than the equivalent murine molecule. This results led to proposing longer linkers between the chains which would contain flexible residues (e.g. alanine or glycine) and polar residues (e.g. serine and threonine). These constructs make up the first class of linkers. To inhibit the continuation of secondary structure across the linker, prolines were added to bracket the linkers. These prolines are known to inhibit the formation of alpha helices and beta sheets. These linkers make up the second class of linkers disclosed here.

Next, flexible regions present in the human MHC and in the murine MHC could be used to make a linker by extending the region of interest and ligating the ends together. These are the third class of linkers. Finally, a combination of these types of linkers could also be used. These are the fourth class of linkers.

### Example 4

### Human MHC Class II Single Chain Molecule with Murine Linkers

Another linker has been suggested based on a combination of murine and human MHC class II single chain molecules. This fusion would incorporate linker residues from the functional murine single chain MHC class II molecule and the alpha and beta chains of the human molecule. The protein sequence of the single chain molecule is provided below (the linker residues from the mouse construct are in bold):

MGDTGRSFTLASSETGVGASGGGGSGGGGDTRPRFLEQVKHECHFFNGTERVRF
LDRYFYHQEEYVRFDSDVGEYRAVTELGRPDAEYWNSQKDLLEQKRAAVDTYC
RHNYGVGESFTVLRRLGGEDDEADHHVIIQAEFYLNPDQSGEFMFDFDGDEIFH
VDMAKKETVWRLEEFGRFASFEAQGALANIAVDKANLEIMTKRSNYTPITN*

Further MHC class II hybrid single chain molecules may be designed by fusing other portions of the alpha chain and the beta chain together using linkers as described elsewhere. A properly folded molecule may be obtained by putting appropriate linkers between portions of the human MHC class II which are proximal to each other as determined by visual inspection of the atomic coordinates of residues of the native MHC available in the publicly accessible protein structure database. These structures would predict possible fusion proteins which covalently attach any part of the beta chain between residues 82 to 123 or between residues 148 to 164 to portions of the alpha chain such as the N-terminal residues, residues 79 to 84, or 92 to 106. The numbering system of residues in this example corresponds to those found in the coordinates of the structure described in: DESSEN, et al. Immunity 7:473 (1997). Other, homologous residues could be used to create equivalent constructs for genotypic and allelic variants of these molecules e.g. equivalent residues in DR2 or such. DNAs for such hybrids would be prepared and expressed in a recombinant expression system by someone skilled in the art and could be assayed for structure and function in appropriate assays.

### Example 5

### Use of CD4 Binding Site(s) of MHC Class II Molecules as Linkers for the Production of Bioactive Recombinant MHC Class II:Peptide Complexes

HLA class II molecules present antigenic peptides to the T cell receptor of the CD4+ T lymphocytes and interact with CD4 during the antigen recognition process. Native MHC class II-peptide complexes have been shown to bind to MHC class II restricted and antigen specific TCRs on a particular T cell and induce T cell nonresponsiveness. It is proposed that the CD4 binding site is important in the docking of MHC class II-peptide complex with the TCR and induce nonresponsiveness. Since the binding of CD4 to MHC class II-peptide is important in antigen presentation and/or induction of T cell nonresponsiveness, it is proposed that recombinant MHC class II-peptide molecules (truncated or whole) containing CD4 binding site will be biologically active. Furthermore, a polypeptide fragment from MHC class II which binds the CD4, when used as a linker in preparation of MHC class II-peptide truncated molecules, provides resulting recombinant molecules that will be biologically active.

The following describes the concept of different linkers. Peptide is attached to a linker 1 (L1) which is attached to N-terminus of β1 domain that is linked to L2. L2 is linked to L3, which in turn is linked to N-terminus of α1 chain of MHC class II. Here L2 represents the human CD4 binding sequences. It should be noted that L2 could also be directly linked to N-terminus of α1 domain by completely deleting L3. Specific examples of L1 and L3 are given in a examples. The sequences of L2 are given below. These sequences are applicable to most of the DR-Peptide molecules.

RNGQEEKAGVVSTGLI,

RNGQETKAGVVSTGLI,

YNQQEEKAGGVSTGLI,

FRNGQEEKAGVVSTGLI,

FRNGQETKAGVVSTGLI,

FYNQQEEKAGGVSTGLI,

and

LNGQEEKAGMVSTGLI.

### Example 6

### I-As MBP.β1β2α1α2.Cκ Construct and Activity

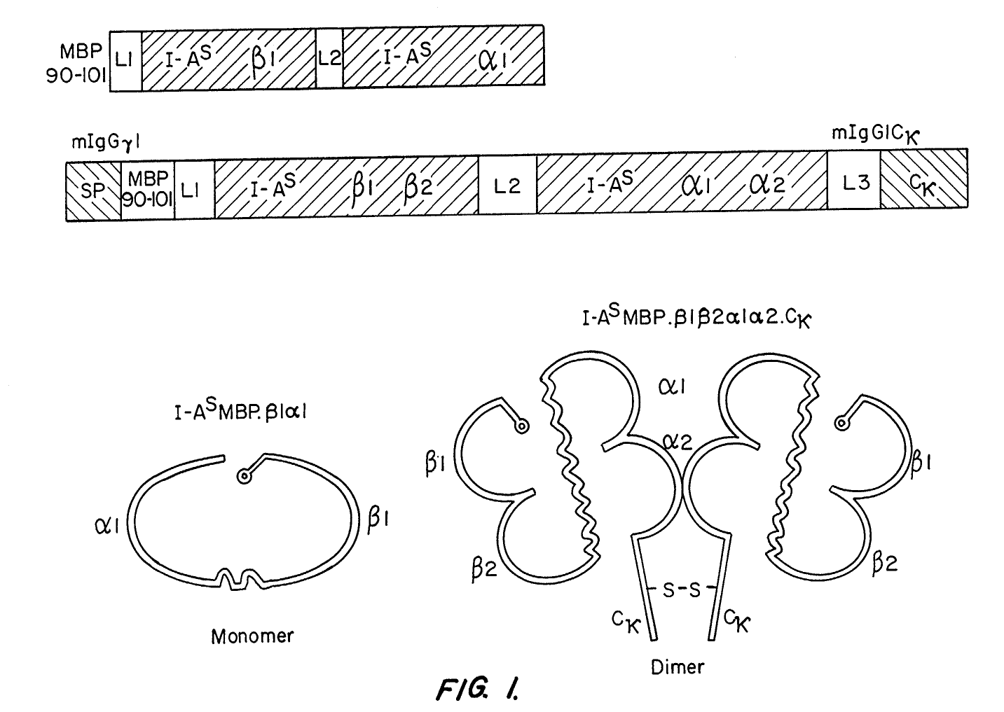

The “full anergix” single chain molecule, mouse I-As MBP.β1β2α1α2.Cκ, was generated by overlap PCR using standard methodology. The molecules was expressed in 293T cells and baculoviral cells according to standard methodology, and purified according to standard methodology using affinity chromatography using goat-anti mouse antibodies. The structure of I-As MBP.β1β2α1α2.Cκ is shown in FIG. 1.

The amino acid sequence of the I-As MBP.Pβ1β2α1α2.Cκ shown in FIG. 1 is as follows:

METDTLLLWVLLLWVPGSTGDFKNIVTPRTPPPASGGGGSGGGGDSERHFVFQF
KGECYFTNGTQRIRSVDRYIYNREEYLRFDSDVGEYRAVTELGRPDPEYYNKQY
LEQTRAELDTVCRHNYEGVETHTSLRRLEQPNVVISLSRTEALNHHNTLVCSVTD
FYPAKIKVRWFRNGQEETVGVSSTQLIRNGDWTFQVLVMLEMTPRRGEVYTCH
VEHPSLKSPITVEWTSGGGGSGGGGSGGGGSGGGGSSSEDDIEADHVGVYGTTV
YQSPGDIGQYTHEFDGDEWFYVDLDKKETIWMLPEFGQLTSFDPQGGLQNIATG
KYTLGILTKRSNSTPATNEAPQATVFPKSPVLLGQPNTLICFVDNIFPPVINITWLR
NSKSVTDGVYETSFLVNRDHSFHKLSYLTFIPSDDDIYDCKVEHWGLEEPVLKHW
ASGGGGSGGGGADAAPTVSIFPPSSEQLTSGGASVVCFLNNFYPKDINVKWKIDG
SERQNGVLNSWTDQDSKDSTYSMSSTLTLTKDEYERHNSYTCEATHKTSTSPIVK
SFNRNEC

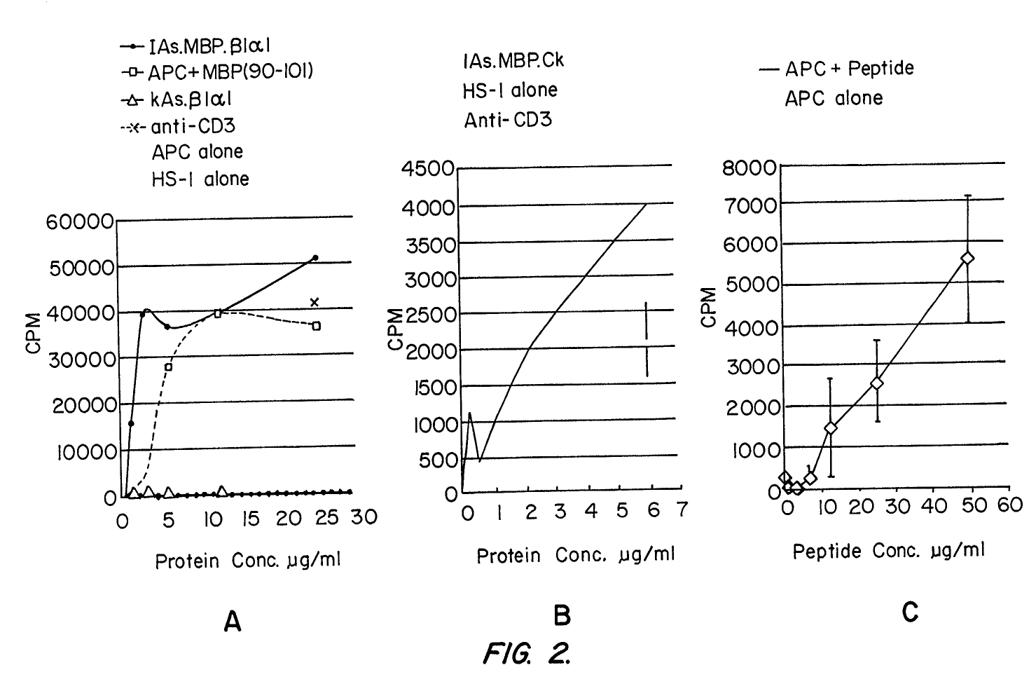

The in vitro activity of recombinant I-As MBP.β1β2α1α2.Cκ was tested using MBP90-101 specific IAs restricted mouse T cell clone HS1. This clone was prepared by the immunization of SJL mice with the MBP90-101 peptide, followed by cloning out CD4+T cells by limited dilution techniques. These cells were maintained by stimulation every 10 days with irradiated SJF splenocytes and PBP90-101 peptide. The T cells are activated by a combination of soluble recombinant I-As MBP.β1β2α1α2.Cκ and plate bound anti-CD28 antibody. T cell activation was assayed by ³H-thymidine incorporation according to standard methodology. FIG. 2 shows the results of this assay.

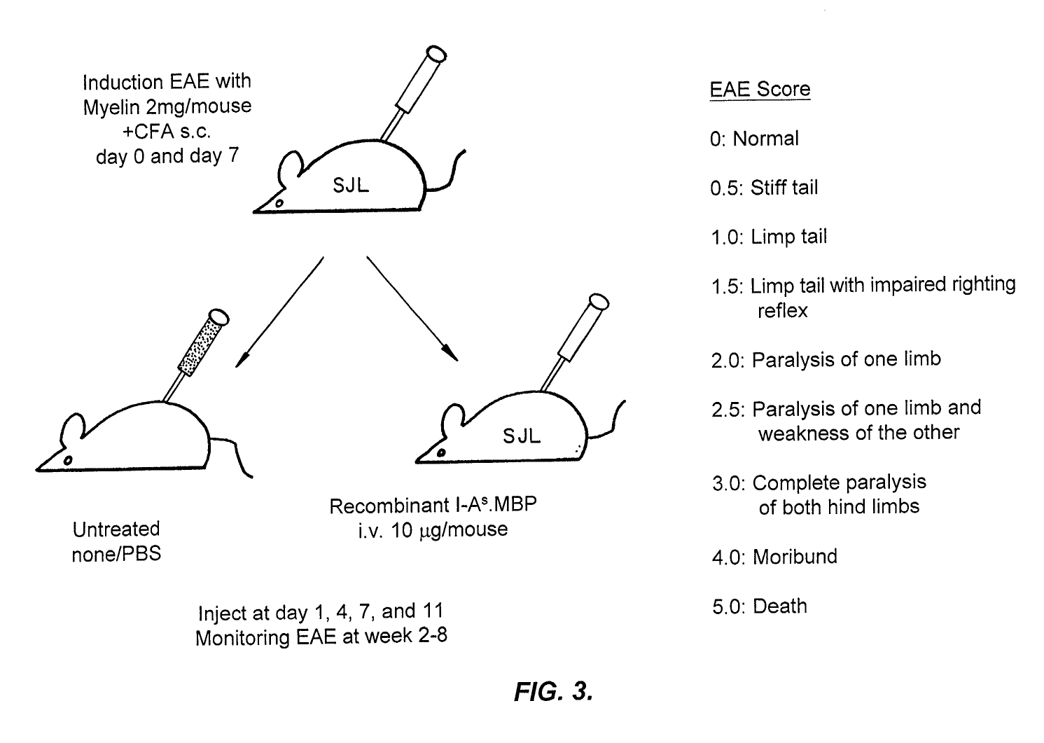

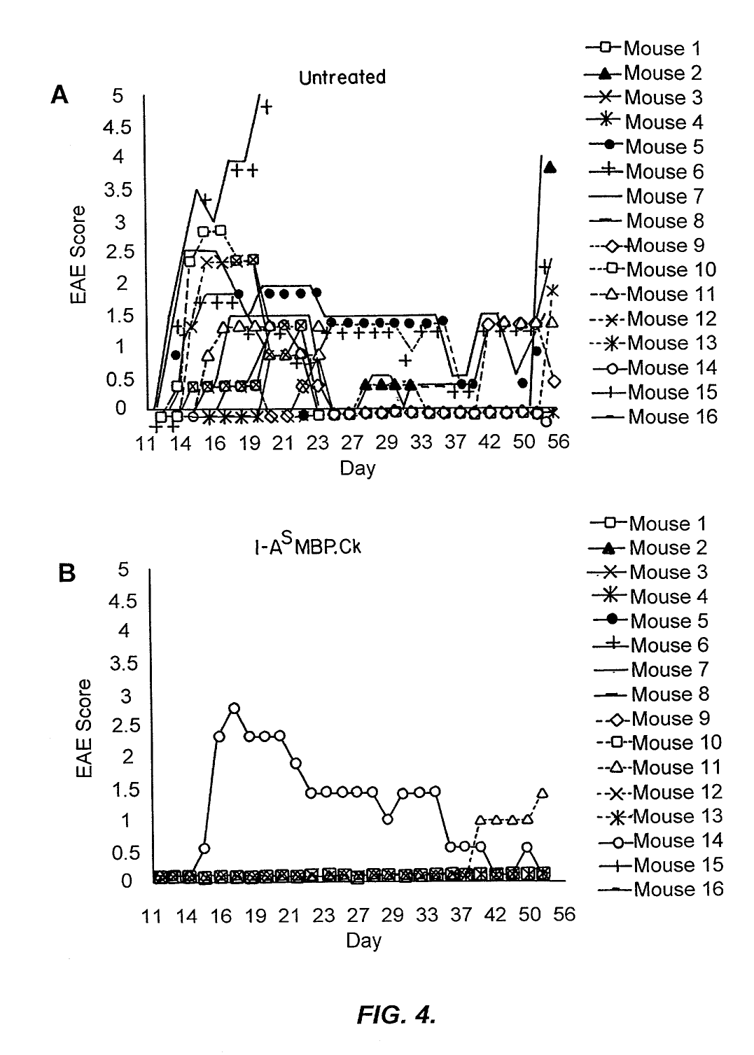

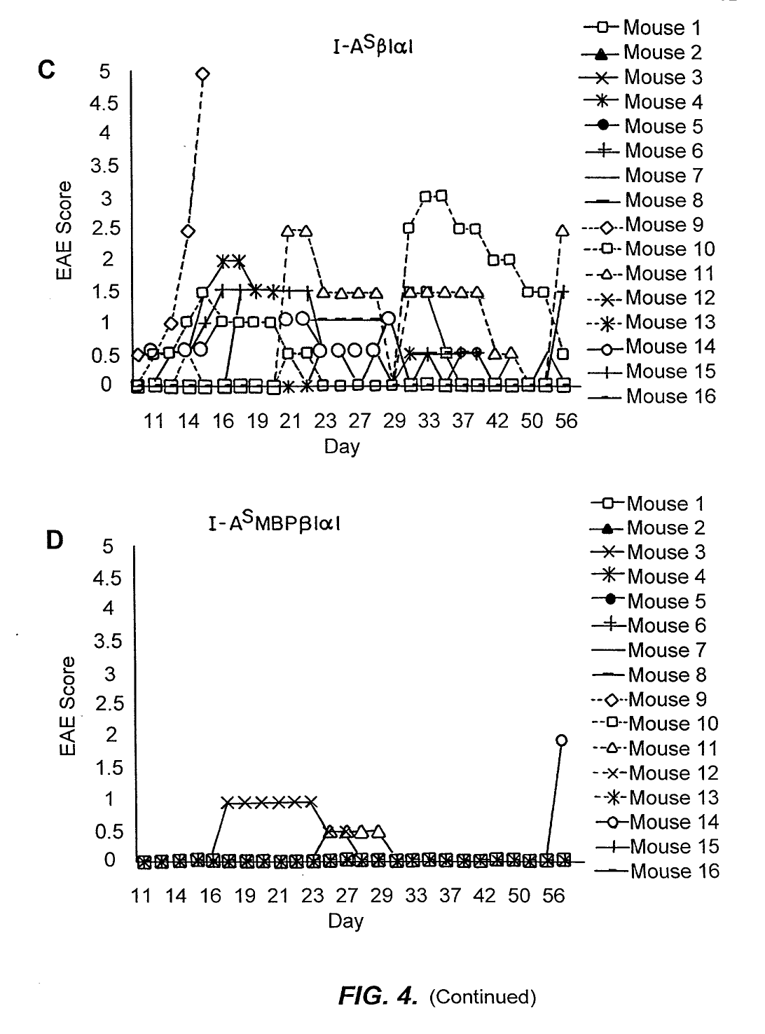

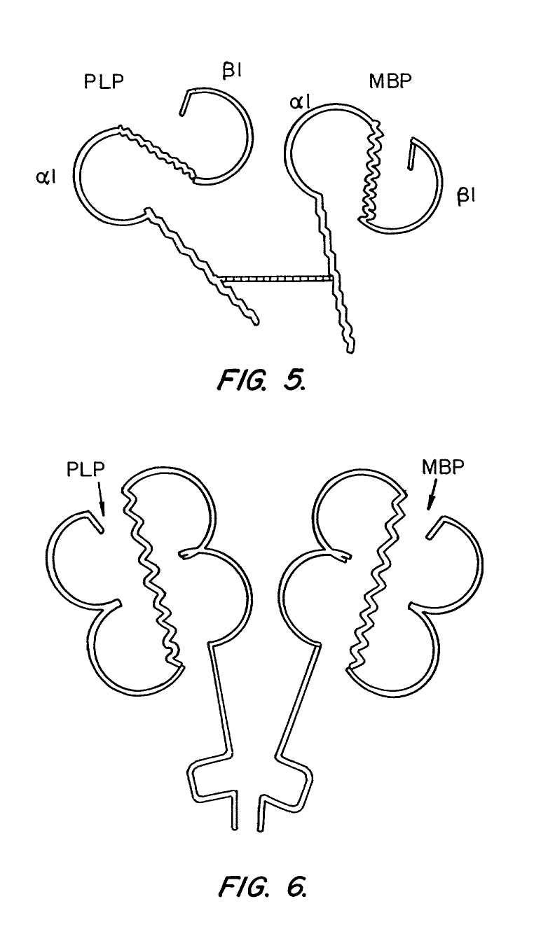

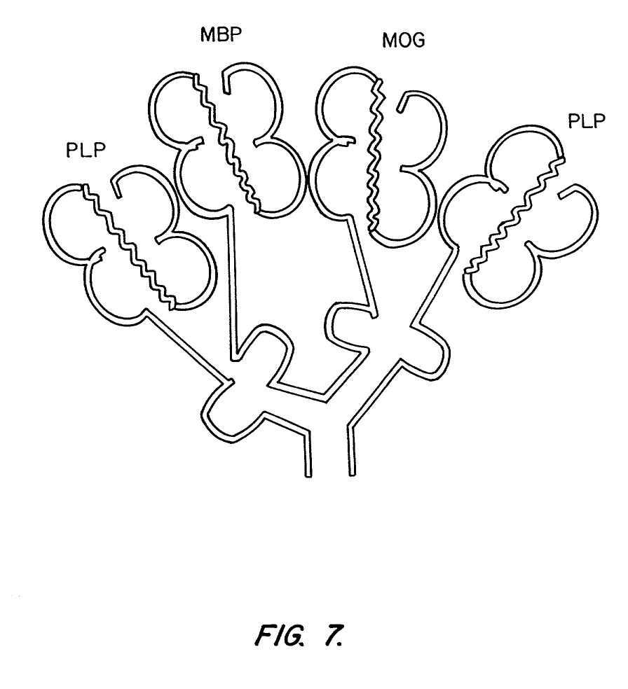

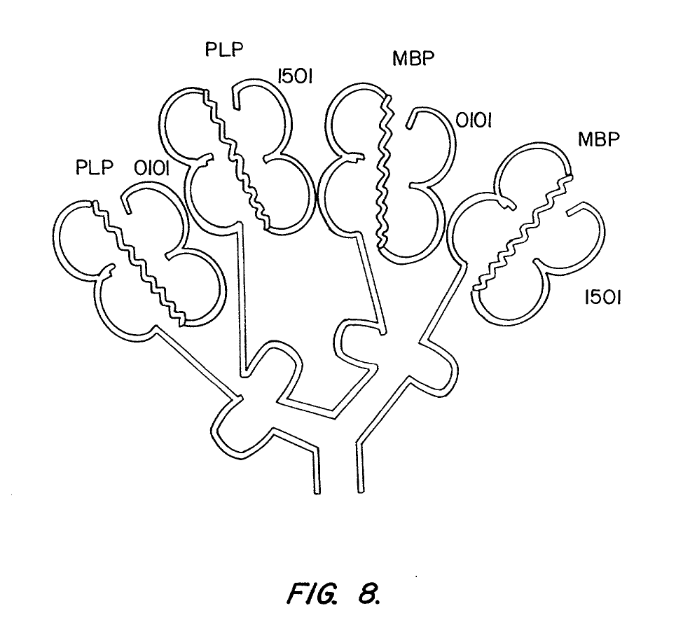

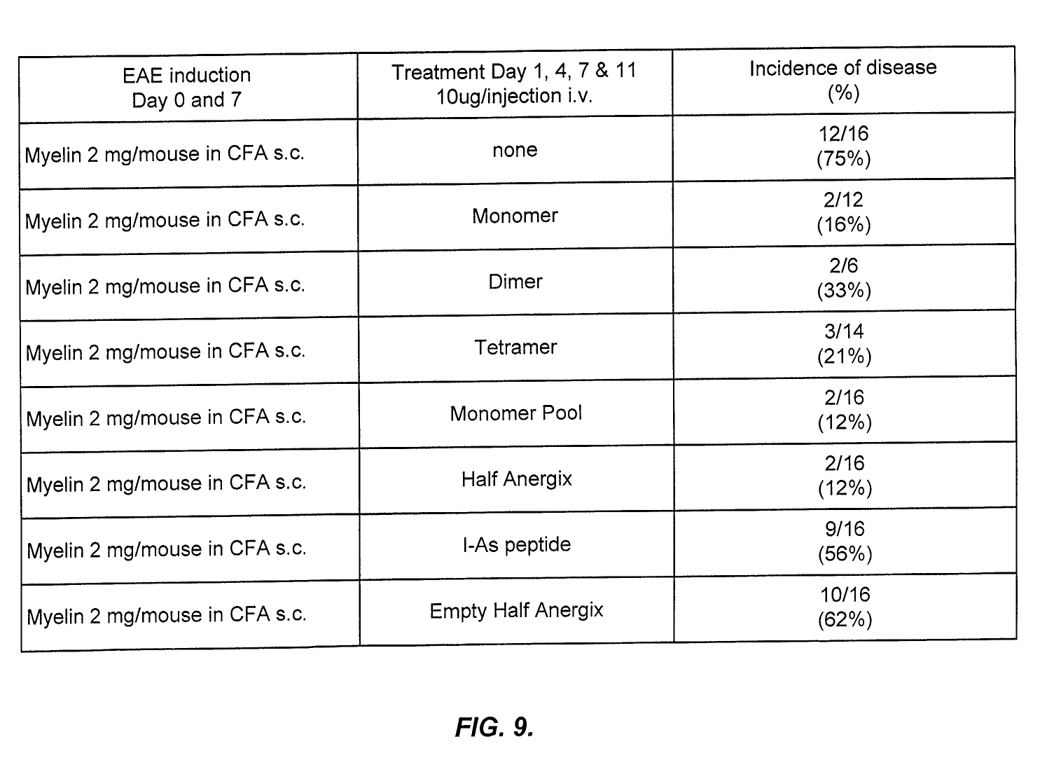

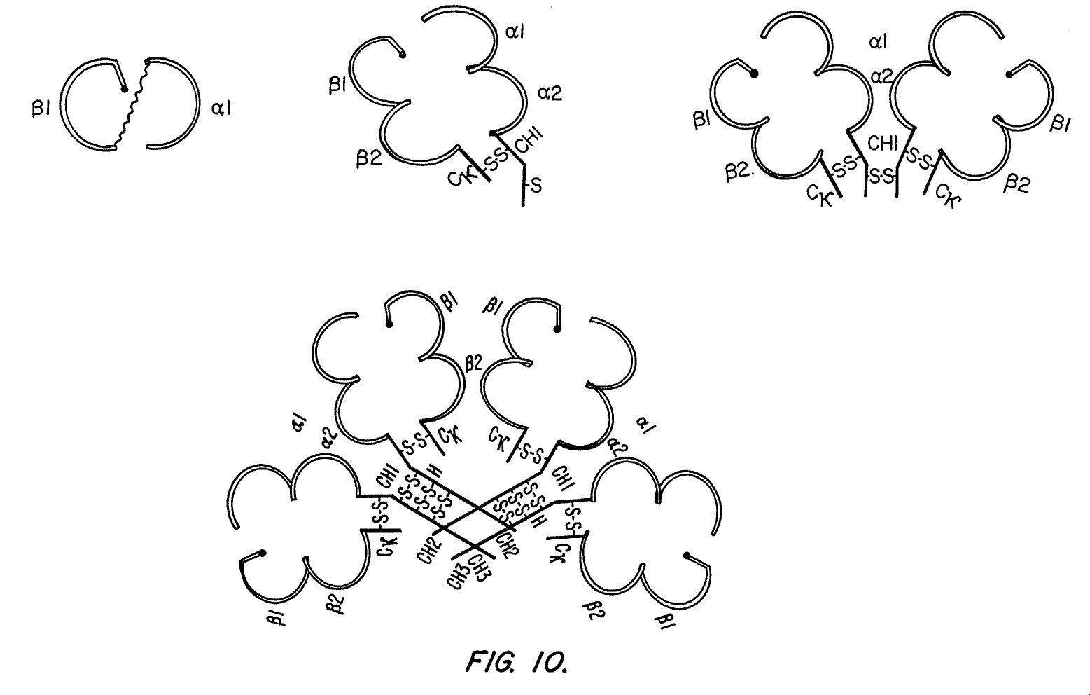

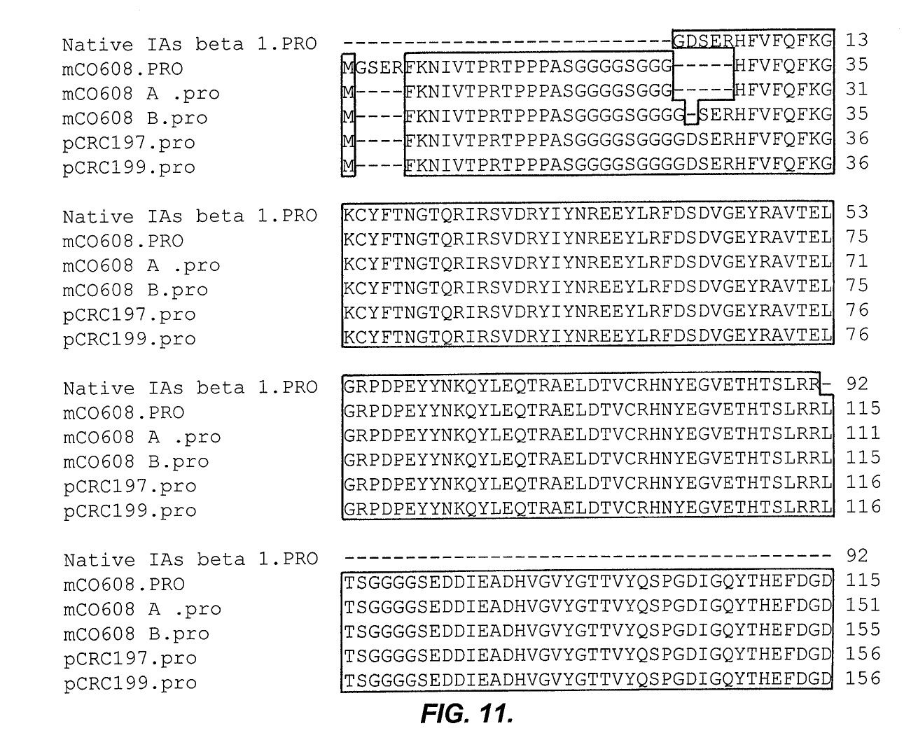

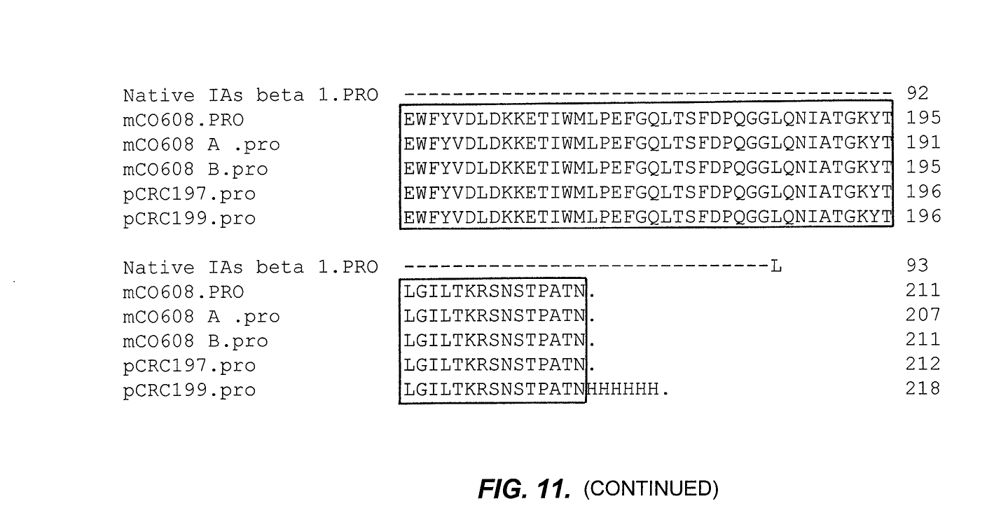

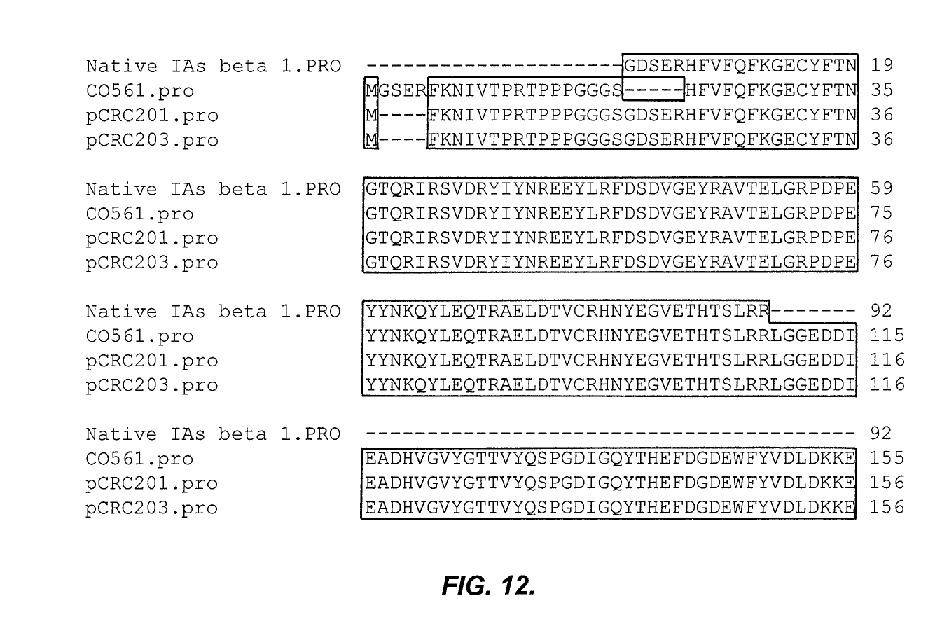

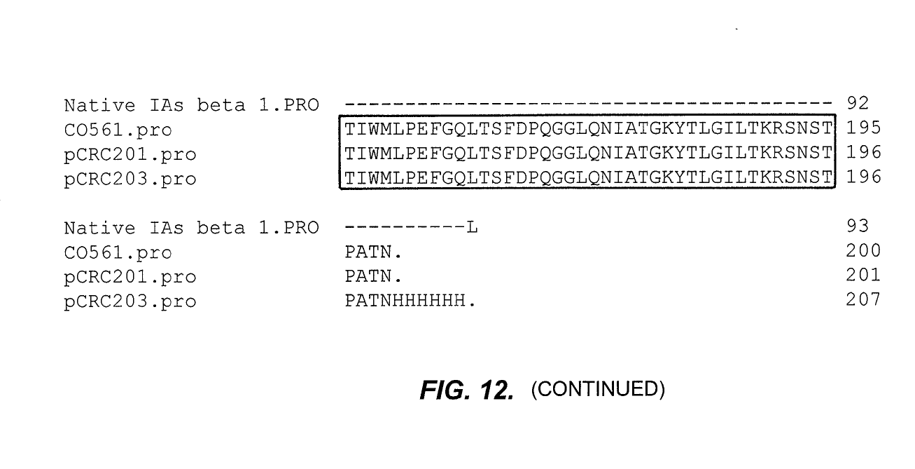

MS is a T cell dependent autoimmune disease caused by localized demyelination in the central nervous system. Experimental autoimmune encephalomyelitis is a accepted animal model of MS. The following results demonstrate that administration of I-As MBP.β1β2α1α2.Cκ reduces the incidence and severity of EAE. EAE was induced according to standard methodology according to the myelin model. Ten μg of recombinant I-As MBP.β1β2α1α2.Cκ was given intravenously in 100 μl of PBS at days 1, 4, 7, and 11 after disease induction. 12 days after immunization, animals are observed daily for the onset of neurological dysfunction. Disease is graded by trained technicians according to standard methods (see FIG. 3). Mice are followed for up to 60-70 days. The data shown in FIG. 4 demonstrate that administration of the recombinant I-As MBP.β1β2α1α2.Cκ significantly reduced the incidence of myelin-induced EAE in SJL mice. In one experiment, 11 out of 20 mice developed EAE in the untreated group (55%), whereas only 2 out of 21 animal showed sign of the disease in the treated group (16.6%). Similarly, in another experiment, 12 out of 16 mice developed EAE in the untreated group (75%), while only 2 out of 16 developed EAE in the treated group (12.5%).

Histological examination from SJL mice in the EAE model studies: spinal cords were removed, fixed in formalin solution and embedded in paraffin. Sections were cut, stained with hematoxylin, eosin and graded for inflammatory lesions. A. Section of spinal cord from untreated mouse, score=2.5; B. Section of spinal cord from mouse treated with the recombinant I-AS.MBP.Ck, score=0; C. Section of spinal cord from mouse treated with the recombinant I-AS.β1α1 without antigenic peptide fusion, score=2.0; D. Section of spinal cord from mouse treated with the recombinant I-AS.MBP.β1α1, score=0.5. The histology score for each section is marked.

### Example 7

### Functional Human Anergix Molecules Optimized for E. coli Expression with E. coli Codons

Two human single chain MHC class II molecules (CO528-AC and CO608-AC) have been optimized for E. Coli expression using “artificial codons,” e.g., preferred E. coli codons encoding the mammalian protein. CO528-AC and CO608-AC were made according to standard PCR overlap technology.

### Example 7

### Recombinant MHC Class II IAs.MBP.Ig Multimeric Complexes

Recombinant MHC class II (IAs)-peptide-Ig fusion complexes were constructed by fusing the mIgG leader, MBP 90-101, or MBP 1-14 (as a control) to IAs single chain (b1b2a1a2), and mIgG.Ck, mIgG.CH1.H, mIgG.CH1.H.CH2, or mIgG.CH1.H.CH2.CH3 with flexible linkers, according to standard methodology. The recombinant IAs fusion proteins were expressed in both mammalian and insect cells and detected by western analysis and ELISA. The overexpressed and secreted recombinant IAs fusion proteins from both human 293 cell cultures or from insect culture medium were purified by affinity chromatography. The purified dimeric and tetrameric recombinant IAs proteins have in vitro biological activity as assayed using an antigen-specific mouse T cell clone. The in vivo activity of the recombinant IAs fusion proteins were studied with the experimental autoimmune encephalomyelitis (EAE) model using susceptible SJL mice. In these EAE studies, recombinant IAs fusion protein was delivered on days 1, 4, 7, and 11 by I.V. injections after induction of the disease with myelin. The animals were then examined for neurological dysfunction. The results indicate that treatment with the recombinant IAs fusion proteins prevents mortality and significantly reduces paralysis induced by myelin homogenate in CFA. In conclusion, these studies suggest that the recombinant MHC class II fusion protein has therapeutic benefit as antigen-specific drugs for the treatment of autoimmune diseases.

### Example 8

### Synthesis of Mouse Model Equivalent of CO608

Four forms of the murine MHC Class II IAs β1α1 with MBP peptide linkers analogous to CO608 human were made. These constructs can be used, e.g., as murine clinical control for human 608. mCO608 (mouse CO608, lacking the first five amino acids, GDSER, as compared to native beta 1 domain), MCO608-A (same as m608 except lacking four amino acids, GSER, after the methionine as compared to m608), B. megaterium-mCO608-A (expressed in Bacillus megaterium), and mCO608-B (same as m608 except lacking first four amino acids, GSER, after the methionine as compared to m608; also lacking second amino acid, D, as compared to native beta 1 domain).

To make mCO608, the upstream linker of mouse CO521 (GGGS) was replaced with the human CO608 linker (ASGGGGSGGG) and the downstream linker of mouse CO521 (GG) was replaced with the downstream linker of CO608 (TSGGGGS), using PCR according to standard methodology. mCO608-A and m608-B were made from mCO608 using PCR according to standard methodology.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A multimeric complex comprising a first recombinant single chain MHC class II molecule and a second recombinant single chain MHC class II molecule, wherein the first and the second single chain MHC class II molecule each comprise an α1 domain and a β1 domain linked via an amino acid linker and a multimerization domain, and wherein the first and the second single chain MHC class II molecule are linked via the multimerization domain to form a multimeric complex.

2. The multimeric complex of claim 1, wherein the first and the second single chain MHC class II molecules are each linked to an antigenic peptide via an amino acid linker.

3. The multimeric complex of claim 2, wherein the peptides are the same.

4. The multimeric complex of claim 2, wherein the peptides are from the same antigen.

5. The multimeric complex of claim 2, wherein the peptides are from different antigens.

6. The multimeric complex of claim 2, wherein the peptides are selected from the group consisting of MBP83-102Y83, PLP 40-60, PLP 89-106, PLP 95-117, and PLP 185-206.

7. The multimeric complex of claim 1, wherein the first and the second MHC class II molecules are from the same MHC class II allele.

8. The multimeric complex of claim 1, wherein the first and the second MHC class II molecules are from different MHC class II alleles.

9. The multimeric complex of claim 7 or 8, wherein the alleles are selected from the group consisting of DRB1*1501 and DRB5*0101.

10. The multimeric complex of claim 7 or 8, wherein the first and the second single chain MHC class II molecules are each linked to an antigenic peptide via an amino acid linker.

11. The multimeric complex of claim 10, wherein the peptides are the same.

12. The multimeric complex of claim 10, wherein the peptides are from the same antigen.

13. The multimeric complex of claim 10, wherein the peptides are from different antigens.

14. The multimeric complex of claim 10, wherein the peptides are selected from the group consisting of MBP83-102Y83, PLP 40-60, PLP 89-106, PLP 95-117, and PLP 185-206.

15. The multimeric complex of claim 1, wherein the first and second MHC class II molecules are from a human.

16. The multimeric complex of claim 1, wherein the multimerization domains are covalently linked.

17. The multimeric complex of claim 1, wherein the multimerization domains are non-covalently linked.

18. The multimeric complex of claim 1, wherein the multimerization domain is a leucine zipper domain.

19. The multimeric complex of claim 1, wherein the multimerization domain is an immunoglobulin domain.

20. The multimeric complex of claim 19, wherein the immunoglobulin domain is a light chain constant domain or a heavy chain constant domain.

21. The multimeric complex of claim 1, wherein the first or the second MHC class II molecule is selected from the group consisting of I-As MBP.β1β2α1α2.Cκ I-As MBP.β1β2α1α2.CH1.H, I-As MBP.β1β2α1α2.CH1.H. CH2, and I-As MBP.β1β2α1α2.CH1.H.CH2.CH3.

22. The multimeric complex of claim 1, further comprising a third and a fourth MHC class II molecule, wherein the third and the fourth MHC class II molecule each comprise an α1 domain and a β1 domain linked via an amino acid linker and a multimerization domain, and wherein the first, second, third and fourth single chain MHC class II molecules are linked via the multimerization domain to form a multimeric complex.

23. The multimeric complex of claim 1, wherein the first and the second MHC class II molecules comprise β1 β2 domains and α1 α2 domains linked via an amino acid linker.

24. The multimeric complex of claim 1 or 2, wherein the linker is selected from the group consisting of GG, GGGG, GGGGS, GGGSG, ASGGGSGGG, TSGGGGSGGGGSSS, GSPGGGGSGGGPGS, GSPPGGPPGS, GSPGGGGPGS, TSGGGGS, SGGSGGS, FDAPSPLP, and VYPEVTV.

25. The multimeric complex of claim 1 or 2, wherein the linker is from a CD4 molecule.

26. The multimeric complex of claim 25, wherein the linker is selected from the group consisting of RNGQEEKAGVVSTGLI ,RNGQETKAGVVSTGLI, YNQQEEKAGGVSTGLI, FRNGQEEKAGVVSTGLI, FRNGQETKAGVVSTGLI, FYNQQEEKAGGVSTGLI, and LNGQEEKAGMVSTGLI.

27. The multimeric complex of claim 1, wherein the first or the second MHC class II molecule is selected from the group consisting of CO523, CO543, CO563, CO567, CO580, CO581, CO582, CO583, CO584, CO585, CO586, CO587, CO588, CO589, CO590, CO591, CO592, CO593, CO594, CO595, CO596, CO597, and CO608.

28. The multimeric complex of claim 1, wherein the first and the second MHC class II molecule are independently selected from the group consisting of CO523, CO543, CO563, CO567, CO580, CO581, CO582, CO583, CO584, CO585, CO586, CO587, CO588, CO589, CO590, CO591, CO592, CO593, CO594, CO595, CO596, CO597, and CO608.

29. The multimeric complex of claim 1, wherein the first MHC class II or the second MHC class II molecule is encoded by a nucleic acid that codons optimized for prokaryotic expression.

30. The multimeric complex of claim 29, wherein the prokaryote is E. coli.

31. The multimeric complex of claim 29, wherein the nucleic acid encoding the first or the second MHC class II molecule is selected from the group consisting of CO528-AC and CO608-AC.

32. A pharmaceutical composition comprising the multimeric complex of claim 2.

33. The pharmaceutical composition of claim 32, further comprising an adjuvant.

34. A method of treating autoimmune disease in a subject, the method comprising administering an immunogenically effective amount of a pharmaceutical composition comprising the multimeric complex of claim 2.

35. The method of claim 34, wherein the subject is a human.

36. A recombinant nucleic acid encoding a single chain MHC class II molecule comprising an α1 domain and a β1 domain linked via an amino acid linker, wherein the linker is selected from the group consisting of GG, GGGG, GGGGS, GGGSG, ASGGGSGGG, TSGGGGSGGGGSSS, GSPGGGGSGGGPGS, GSPPGGPPGS, GSPGGGGPGS, TSGGGGS, SGGSGGS, FDAPSPLP, and VYPEVTV.

37. The nucleic acid of claim 36, wherein the MHC class II molecule is selected from the group consisting of CO523, CO543, CO563, CO567, CO580, CO581, CO582, CO583, CO584, CO585, CO586, CO587, CO588, CO589, CO590, CO591, CO592, CO593, CO594, CO595, CO596, CO597, and CO608.

38. A recombinant nucleic acid encoding a single chain MHC class II molecule comprising an α1 domain and a β1 domain linked via an amino acid linker, wherein the linker is wherein the linker is from a CD4 molecule.

39. The nucleic acid of claim 38, wherein the linker is selected from the group consisting of RNGQEEKAGVVSTGLI ,RNGQETKAGVVSTGLI, YNQQEEKAGGVSTGLI, FRNGQEEKAGVVSTGLI, FRNGQETKAGVVSTGLI, FYNQQEEKAGGVSTGLI, and LNGQEEKAGMVSTGLI.

40. A recombinant nucleic acid encoding a single chain MHC class II molecule selected from the group consisting of CO528-AC and CO608-AC.

41. A recombinant nucleic acid encoding a single chain MHC class II molecule selected from the group consisting I-As MBP.β1β2α1α2.Cκ, I-As MBP.β1β2α1α2.CH1.H, I-As MBP.β1β2α1α2.CH1.H.CH2, and I-As MBP.β1β2α1α2.CH1.H.CH2.CH3.

42. A recombinant nucleic acid encoding a single chain MHC class II molecule selected from the group consisting CO523, CO543, CO563, CO567, CO580, CO581, CO582, CO583, CO584, CO585, CO586, CO587, CO588, CO589, CO590, CO591, CO592, CO593, CO594, CO595, CO596, CO597, and CO608.


SEQUENCE LISTING (129 sequences)

SEQ ID NO: 1 - DNA, 558 bp, Artificial Sequence
  Description of Artificial SequenceCO602 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgctgcgccgactcggaggtgaagatgacgaggcagatcaccatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 2 - DNA, 558 bp, Artificial Sequence
  Description of Artificial SequenceCO601 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggaggtggaagcggcggaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 3 - DNA, 555 bp, Artificial Sequence
  Description of Artificial SequenceCO600 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggaggtggaggcagcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 4 - DNA, 552 bp, Artificial Sequence
  Description of Artificial SequenceCO599 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggaggtggaggcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 5 - DNA, 561 bp, Artificial Sequence
  Description of Artificial SequenceCO594 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgagtctaccctgaggtaactgtcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 6 - DNA, 564 bp, Artificial Sequence
  Description of Artificial SequenceCO592 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgattcgacgcacctagcccactcccaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 7 - DNA, 561 bp, Artificial Sequence
  Description of Artificial SequenceCO591 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaagtggcggtagtggcggtagtatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 8 - DNA, 561 bp, Artificial Sequence
  Description of Artificial SequenceCO590 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaactagtggtggcggtggcagcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 9 - DNA, 570 bp, Artificial Sequence
  Description of Artificial SequenceCO589 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggctctcctggaggtggaggtcctggatctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 10 - DNA, 570 bp, Artificial Sequence
  Description of Artificial SequenceCO588 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggctctcctcctggtggaccacctggatctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 11 - DNA, 582 bp, Artificial Sequence
  Description of Artificial SequenceCO587 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgagggagtccaggcggtggcgggagcggcggtggtcctgggagtatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 12 - DNA, 582 bp, Artificial Sequence
  Description of Artificial SequenceCO586 single chain recombinant MHC class II molecule
atgggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaactagtggtggcggtggcagcggcggtggtggttcctcgagtatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 13 - protein, 185 aa, Artificial Sequence
  Description of Artificial SequenceCO602 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVLRRLGGEDDEADHHVIIQAEFYLNPDQSGEFMFDFDGDEIFHVDMAKKETVWRLEEFGRFASFEAQGALANIAVDKANLEIMTKRSNYTPITN

SEQ ID NO: 14 - protein, 185 aa, Artificial Sequence
  Description of Artificial SequenceCO601 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGGGSGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 15 - protein, 184 aa, Artificial Sequence
  Description of Artificial SequenceCO600 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGGGGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 16 - protein, 183 aa, Artificial Sequence
  Description of Artificial SequenceCO599 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGGGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 17 - protein, 186 aa, Artificial Sequence
  Description of Artificial SequenceCO594 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 18 - protein, 187 aa, Artificial Sequence
  Description of Artificial SequenceCO592 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRFDAPSPLPIKEEHVIIQAEFYLNPDQSGEFMFDFDGDEIFHVDMAKKETVWRLEEFGRFASFEAQGALANIAVDKANLEIMTKRSNYTPITN

SEQ ID NO: 19 - protein, 186 aa, Artificial Sequence
  Description of Artificial SequenceCO591 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRSGGSGGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 20 - protein, 186 aa, Artificial Sequence
  Description of Artificial SequenceCO590 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRTSGGGGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 21 - protein, 189 aa, Artificial Sequence
  Description of Artificial SequenceCO589 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGSPGGGGPGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 22 - protein, 189 aa, Artificial Sequence
  Description of Artificial SequenceCO588 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGSPPGGPPGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 23 - protein, 193 aa, Artificial Sequence
  Description of Artificial SequenceCO587 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGSPGGGGSGGGPGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 24 - protein, 193 aa, Artificial Sequence
  Description of Artificial SequenceCO586 single chain recombinant MHC class II molecule
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRTSGGGGSGGGGSSSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 25 - DNA, 639 bp, Artificial Sequence
  Description of Artificial SequenceCO598 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgctgcgccgactcggaggtgaagatgacgaggcagatcaccatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 26 - DNA, 639 bp, Artificial Sequence
  Description of Artificial SequenceCO597 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggaggtggaagcggcggaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 27 - DNA, 636 bp, Artificial Sequence
  Description of Artificial SequenceCO596 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggaggtggaggcagcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 28 - DNA, 633 bp, Artificial Sequence
  Description of Artificial SequenceCO595 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggaggtggaggcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 29 - DNA, 642 bp, Artificial Sequence
  Description of Artificial SequenceCO593 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgagtctaccctgaggtaactgtcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 30 - DNA, 645 bp, Artificial Sequence
  Description of Artificial SequenceCO585 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgattcgacgcacctagcccactcccaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 31 - DNA, 642 bp, Artificial Sequence
  Description of Artificial SequenceCO584 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaagtggcggtagtggcggtagtatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 32 - DNA, 642 bp, Artificial Sequence
  Description of Artificial SequenceCO583 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaactagtggtggcggtggcagcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 33 - DNA, 651 bp, Artificial Sequence
  Description of Artificial SequenceCO582 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggctctcctggaggtggaggtcctggatctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 34 - DNA, 651 bp, Artificial Sequence
  Description of Artificial SequenceCO581 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaggctctcctcctggtggaccacctggatctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 35 - DNA, 663 bp, Artificial Sequence
  Description of Artificial SequenceCO580 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgagggagtccaggcggtggcgggagcggcggtggtcctgggagtatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 36 - DNA, 663 bp, Artificial Sequence
  Description of Artificial SequenceCO567 single chain recombinant MHC class II molecule
atgggggacaccggtcgcagcttcaccctggcctccagcgagaccggcgtgggcgctagcggagggggcggaagcggcggagggggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaactagtggtggcggtggcagcggcggtggtggttcctcgagtatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 37 - protein, 212 aa, Artificial Sequence
  Description of Artificial SequenceCO598 single chain recombinant MHC class II molecule, human MHC class II single chain molecule with murine linkers
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVLRRLGGEDDEADHHVIIQAEFYLNPDQSGEFMFDFDGDEIFHVDMAKKETVWRLEEFGRFASFEAQGALANIAVDKANLEIMTKRSNYTPITN

SEQ ID NO: 38 - protein, 212 aa, Artificial Sequence
  Description of Artificial SequenceCO597 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGGGSGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 39 - protein, 211 aa, Artificial Sequence
  Description of Artificial SequenceCO596 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGGGGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 40 - protein, 210 aa, Artificial Sequence
  Description of Artificial SequenceCO595 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGGGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 41 - protein, 213 aa, Artificial Sequence
  Description of Artificial SequenceCO593 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 42 - protein, 214 aa, Artificial Sequence
  Description of Artificial SequenceCO585 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRFDAPSPLPIKEEHVIIQAEFYLNPDQSGEFMFDFDGDEIFHVDMAKKETVWRLEEFGRFASFEAQGALANIAVDKANLEIMTKRSNYTPITN

SEQ ID NO: 43 - protein, 213 aa, Artificial Sequence
  Description of Artificial SequenceCO584 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRSGGSGGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 44 - protein, 213 aa, Artificial Sequence
  Description of Artificial SequenceCO583 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRTSGGGGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 45 - protein, 216 aa, Artificial Sequence
  Description of Artificial SequenceCO582 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGSPGGGGPGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 46 - protein, 216 aa, Artificial Sequence
  Description of Artificial SequenceCO581 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGSPPGGPPGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 47 - protein, 220 aa, Artificial Sequence
  Description of Artificial SequenceCO580 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGSPGGGGSGGGPGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 48 - protein, 220 aa, Artificial Sequence
  Description of Artificial SequenceCO567 single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRTSGGGGSGGGGSSSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 49 - DNA, 1680 bp, Artificial Sequence
  Description of Artificial SequenceI- As MBP.beta1beta2alpha1alpha2.Ck single chain molecule
gcggccgccaccatggagacagacacactcctgctatgggtactgctgctctgggttccaggttccactggtgacttcaagaacattgtgacacctcgaacaccacctccagctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagggtacc

SEQ ID NO: 50 - protein, 553 aa, Artificial Sequence
  Description of Artificial SequenceI- As MBP.beta1beta2alpha1alpha2.Ck single chain molecule
METDTLLLWVLLLWVPGSTGDFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 51 - DNA, 600 bp, Artificial Sequence
  Description of Artificial SequenceCO528-AC single chain recombinant MHC class II molecule
atgggggacaccggtcgcagcttcaccctggcctccagcgagaccggcgtgggtggtggcggttcgcgtccacgtttcttggaacaggttaaacatgagtgtcattttttcaatgggacggaacgcgtgcgttttctggatcgttacttttatcaccaagaggaatacgtacgcttcgacagcgatgtgggcgaatatcgtgcggtcacggaactgggtcgtcctgatgccgaatactggaacagtcagaaggacttactggagcagaaacgtgcagcggtggatacctattgccgccacaattacggcgttggtgaaagcttcacagtccagcgtcgcggtggcatcaaagaagagcatgtgattatccaggcggaattctatctgaatccggatcaatcgggcgaattcatgtttgacttcgatggtgatgagattttccatgttgatatggcaaagaaagaaacggtctggcgcttagaggaatttggccgctttgcctcgttcgaagctcaaggcgcattggccaacattgctgtggataaagcgaacctggaaatcatgacaaaacgctccaactatactccgattaccaattaa

SEQ ID NO: 52 - DNA, 642 bp, Artificial Sequence
  Description of Artificial SequenceCO608-AC single chain recombinant MHC class II molecule
atgggggacaccggtcgcagcttcaccctggcctccagcgagaccggcgtgggtgcttctggcgggggcggttcgggcggtgggggtgacacccgtccacgtttcttggaacaggttaaacatgagtgtcattttttcaatgggacggaacgcgtgcgttttctggatcgttacttttatcaccaagaggaatacgtacgcttcgacagcgatgtgggcgaatatcgtgcggtcacggaactgggtcgtcctgatgccgaatactggaacagtcagaaggacttactggagcagaaacgtgcagcggtggatacctattgccgccacaattacggcgttggtgaaagcttcacagtccagcgtcgcactagtggtggcggtggctctatcaaagaagagcatgtgattatccaggcggaattctatctgaatccggatcaatcgggcgaattcatgtttgacttcgatggtgatgagattttccatgttgatatggcaaagaaagaaacggtctggcgcttagaggaatttggccgctttgcctcgttcgaagctcaaggcgcattggccaacattgctgtggataaagcgaacctggaaatcatgacaaaacgctccaactatactccgattaccaattaa

SEQ ID NO: 53 - DNA, 600 bp, Artificial Sequence
  Description of Artificial SequenceCO528 single chain recombinant MHC class II molecule
atgggggacaccggaagatcgttcacactcgcatcatcagagacaggagtaggaggaggaggatcgcgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagagggacctcctggagcagaagcgggccgcggtggacacctactgcagacacaactacggggttggtgagagcttcacagtgcagcggcgaggaggtatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 54 - DNA, 630 bp, Artificial Sequence
  Description of Artificial SequenceCO608 single chain recombinant MHC class II molecule
atgggggacacccgcagcttcaccctggcctccagcgagaccggcgtgggcgctagcggagggggcggaagcggcggaggggggccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgaactagtggtggcggtggcagcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaa

SEQ ID NO: 55 - DNA, 642 bp, Artificial Sequence; identical to SEQ ID NO: 32
  Description of Artificial SequenceCO608 variation single chain recombinant MHC class II molecule

SEQ ID NO: 56 - protein, 199 aa, Artificial Sequence
  Description of Artificial SequenceCO528-AC single chain recombinant MHC class II molecule
MGDTGRSFTLASSETGVGGGGSRPRFLEQVKHECHFFNGTERVRFLDRYFYHQEEYVRFDSDVGEYRAVTELGRPDAEYWNSQKDLLEQKRAAVDTYCRHNYGVGESFTVQRRGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 57 - protein, 213 aa, Artificial Sequence; identical to SEQ ID NO: 44
  Description of Artificial SequenceCO608-AC single chain recombinant MHC class II molecule

SEQ ID NO: 58 - protein, 209 aa, Artificial Sequence
  Description of Artificial SequenceCO608 single chain recombinant MHC class II molecule
MGDTRSFTLASSETGVGASGGGGSGGGGPRFLEQVKHECHFFNGTERVRFLDRYFYHQEEYVRFDSDVGEYRAVTELGRPDAEYWNSQKDLLEQKRAAVDTYCRHNYGVGESFTVQRRTSGGGGSIKEEHVIIQAEFYLNPDQSGEFMFDFDGDEIFHVDMAKKETVWRLEEFGRFASFEAQGALANIAVDKANLEIMTKRSNYTPITN

SEQ ID NO: 59 - protein, 211 aa, Artificial Sequence
  Description of Artificial SequenceCO608 variation single chain recombinant MHC class II molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRTSGGGGSIKEEHVIIQAEFYLNPDQSGEFMFDFDGDEIFHVDMAKKETVWRLEEFGRFASFEAQGALANIADKANLEIMTKRSNYTPIT

SEQ ID NO: 60 - protein, 199 aa, Artificial Sequence
  Description of Artificial SequenceCO528 single chain recombinant MHC class II molecule
MGDTGRSFTLASSETGVGGGGSRPRFLEQVKHECHFFNGTERVRFLDRYFYHQEEYVRFDSDVGEYRAVTELGRPDAEYWNSQRDLLEQKRAAVDTYCRHNYGVGESFTVQRRGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 61 - DNA, 2346 bp, Artificial Sequence
  Description of Artificial SequenceIAs MBP 1-14 CH1.H.CH2.CH3 recombinant MHC class II-peptide-Ig fusion complex
gcggccgccaccatggagacagacacactcctgctatgggtactgctgctctgggttccaggttccactggtgacatggcgtcacagaagagaccctcccagaggcacggatccaaggctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatctggtacctgc

SEQ ID NO: 62 - DNA, 1701 bp, Artificial Sequence
  Description of Artificial SequenceIAs MBP 1-14 CH1.H recombinant MHC class II-peptide-Ig fusion complex
gcggccgccaccatggagacagacacactcctgctatgggtactgctgctctgggttccaggttccactggtgacatggcgtcacagaagagaccctcccagaggcacggatccaaggctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtctaaggtacc

SEQ ID NO: 63 - DNA, 2053 bp, Artificial Sequence
  Description of Artificial SequenceIAs MBP 90- 101 CH1.H.CH2 recombinant MHC class II-peptide-Ig fusion complex
gcggccgccaccatggagacagacacactcctgctatgggtactgctgctctgggttccaggttccactggtgacttcaagaacattgtgacacctcgaacaccacctccagctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagccaaaacaacacccccatcagtctatccactggcccctgggtgtggagatacaactggttcctccgtgactctgggatgcctggtcaagggctacttccctgagtcagtgactgtgacttggaactctggctccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctggagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaatagggtaccccga

SEQ ID NO: 64 - DNA, 1707 bp, Artificial Sequence
  Description of Artificial SequenceIAs MBP 90- 101 CH1.H recombinant MHC class II-peptide-Ig fusion complex
gcggccgccaccatggagacagacacactcctgctatgggtactgctgctctgggttccaggttccactggtgacttcaagaacattgtgacacctcgaacaccacctccagctagcgctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagcttaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtctaaggtacc

SEQ ID NO: 65 - DNA, 1686 bp, Artificial Sequence
  Description of Artificial SequenceIAs MBP 1-14 Ck recombinant MHC class II-peptide-Ig fusion complex
gcggccgccaccatggagacagacacactcctgctatgggtactgctgctctgggttccaggttccactggtgacatggcgtcacagaagagaccctcccagaggcacggatccaaggctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagggtacc

SEQ ID NO: 66 - DNA, 2059 bp, Artificial Sequence
  Description of Artificial SequenceIAs MBP 1-14 CH1.H.CH2 recombinant MHC class II-peptide-Ig fusion complex
gcggccgccaccatggagacagacacactcctgctatgggtactgctgctctgggttccaggttccactggtgacatggcgtcacagaagagaccctcccagaggcacggatccaaggctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagccaaaacaacacccccatcagtctatccactggcccctgggtgtggagatacaactggttcctccgtgactctgggatgcctggtcaagggctacttccctgagtcagtgactgtgacttggaactctggctccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctggagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaatagggtaccccga

SEQ ID NO: 67 - DNA, 2343 bp, Artificial Sequence
  Description of Artificial SequenceIAs MBP 90- 101 CH1.H.CH2.CH3 recombinant MHC class II-peptide-Ig fusion complex
gcggccgccaccatggagacagacacactcctgctatgggtactgctgctctgggttccaggttccactggtgacttcaagaacattgtgacacctcgaacaccacctccagctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacgacgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggaggaagcttagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatctggtacctgc

SEQ ID NO: 68 - protein, 678 aa, Artificial Sequence
  Description of Artificial SequenceIAs MBP 1-14 CH1.H.CH2.CH3 recombinant MHC class II-peptide-Ig fusion complex
METDTLLLWVLLLWVPGSTGDMASQKRPSQRHGSKASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIK

SEQ ID NO: 69 - protein, 676 aa, Artificial Sequence
  Description of Artificial SequenceIAs MBP 1-14 CH1.H recombinant MHC class II-peptide-Ig fusion complex
METDTLLLWVLLLWVPGSTGDFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIK

SEQ ID NO: 70 - protein, 562 aa, Artificial Sequence
  Description of Artificial SequenceIAs MBP 90- 101 CH1.H.CH2 recombinant MHC class II-peptide-Ig fusion complex
METDTLLLWVLLLWVPGSTGDFKNIVTPRTPPPASASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSLSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTV

SEQ ID NO: 71 - protein, 555 aa, Artificial Sequence
  Description of Artificial SequenceIAs MBP 90 101 CH1.H recombinant MHC class II-peptide-Ig fusion complex
METDTLLLWVLLLWVPGSTGDMASQKRPSQRHGSKASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 72 - protein, 774 aa, Artificial Sequence
  Description of Artificial SequenceIAs MBP 1- 14 Ck recombinant MHC class II-peptide-Ig fusion complex
TMETDTLLLWVLLLWVPGSTGDMASQKRPSQRHGSKASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 73 - protein, 560 aa, Artificial Sequence
  Description of Artificial SequenceIAs MBP 1- 14 CH1.H.CH2 recombinant MHC class II-peptide-Ig fusion complex
METDTLLLWVLLLWVPGSTGDMASQKRPSQRHGSKASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTV

SEQ ID NO: 74 - protein, 772 aa, Artificial Sequence
  Description of Artificial SequenceIAs MBP 90- 101 CH1.H.CH2.CH3 recombinant MHC class II-peptide-Ig fusion complex
METDTLLLWVLLLWVPGSTGDFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTTCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGSLAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 75 - DNA, 609 bp, Artificial Sequence
  Description of Artificial SequencepCRC203 recombinant MHC class II heterodimer
catatgttcaagaacattgtgacacctcgaacaccacctccaggaggaggatccggagactcggaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttggaggtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatctcgag

SEQ ID NO: 76 - DNA, 614 bp, Artificial Sequence
  Description of Artificial SequencepCRC201 recombinant MHC class II heterodimer
catatgttcaagaacattgtgacacctcgaacaccacctccaggaggaggatccggagactcggaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttggaggtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaattaagcggccgc

SEQ ID NO: 77 - DNA, 642 bp, Artificial Sequence
  Description of Artificial SequencepCRC199 recombinant MHC class II heterodimer
catatgttcaagaacattgtgacacctcgaacaccacctccagctagcggagggggcggaagcggcggagggggagactccgaaaggcatttcgtgttccagtttaaaggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttactagtggtggcggtggcagcgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatctcgag

SEQ ID NO: 78 - DNA, 647 bp, Artificial Sequence
  Description of Artificial SequencepCRC197 recombinant MHC class II heterodimer
catatgttcaagaacattgtgacacctcgaacaccacctccagctagcggagggggcggaagcggcggagggggagactccgaaaggcatttcgtgttccagtttaaaggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttactagtggtggcggtggcagcgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaattaagcggccgc

SEQ ID NO: 79 - DNA, 1698 bp, Artificial Sequence
  Description of Artificial SequencepCRC188 recombinant MHC class II heterodimer
gcggccgccaccatggctctgcagatccccagcctcctcctctcggctgctgtggtggtgctgatggtgctgagcagcccagggactgagggcttcaagaacattgtgacacctcgaacaccacctccagctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagggtacc

SEQ ID NO: 80 - DNA, 1662 bp, Artificial Sequence
  Description of Artificial SequencepCRC187 recombinant MHC class II heterodimer
gcggccgccaccatggctctgcagatccccagcctcctcctctcggctgctgtggtggtgctgatggtgctgagcagcccagggactgagggcgctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggactagtggtggcggtggcagcggcggtggtggttccggtggcggcggttctggcggtggcggttcctcgagtgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggctagcggagggggcggaagcggcggagggggagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagggtacc

SEQ ID NO: 81 - DNA, 1085 bp, Artificial Sequence
  Description of Artificial SequencepCB229 recombinant MHC class II heterodimer
gaattcggtaccaccatggctctgcagatccccagcctcctcctctcggctgctgtggtggtgctgatggtgctgagcagcccagggactgagggcttcaagaacattgtgacacctcgaacaccacctccagctagcggagggggcggaagcggcggagggggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggagggcacagtctgagtctgcccggagcggatccgctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaggcggccgc

SEQ ID NO: 82 - DNA, 1676 bp, Artificial Sequence
  Description of Artificial SequencepCB223 recombinant MHC class II heterodimer
gaattcggtaccatggctctgcagatccccagcctcctcctctcggctgctgtggtggtgctgatggtgctgagcagcccagggactgagggcgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggaacctgagattccagcccccatgtcagaaggatctgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagcggccgc

SEQ ID NO: 83 - DNA, 773 bp, Artificial Sequence
  Description of Artificial SequencepCB212 recombinant MHC class II heterodimer
ccatgggtaagaaacagaccgctgttgcattcgctctggcgctcctggctctttctatgaccccggcgtacgctttcaagaacattgtgacacctcgaacaccacctccagctagcggagggggcggaagcggcggagggggagactccgaaaggcatttcgtgttccagtttaaaggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttggtggcggtggcagcggcggtggtggttccggtggcggcggttctggcggtggcggttccggtggcggtggcagcgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaattaaggtaccggatcc

SEQ ID NO: 84 - DNA, 702 bp, Artificial Sequence
  Description of Artificial SequencepCB214 recombinant MHC class II heterodimer
atgttcaagaacattgtgacacctcgaacaccacctccagctagcggagggggcggaagcggcggagggggagactccgaaaggcatttcgtgttccagtttaaaggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttggtggcggtggcagcggcggtggtggttccggtggcggcggttctggcggtggcggttccggtggcggtggcagcgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaattaaggtaccggatcc

SEQ ID NO: 85 - DNA, 1013 bp, Artificial Sequence
  Description of Artificial SequencepCB220 recombinant MHC class II heterodimer
gaattcggtaccatggctctgcagatccccagcctcctcctctcggctgctgtggtggtgctgatggtgctgagcagcccagggactgagggcgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagtaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggaacctgagattccagcccccatgtcagaaggatccgccaaaacaacagccccatcggtctatccactggcccctgtgtgtggagatacaactggctcctcggtgactctaggatgcctggtcaagggttatttccctgagccagtgaccttgacctggaactctggatctctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtgctgcataggcggccgc

SEQ ID NO: 86 - protein, 206 aa, Artificial Sequence
  Description of Artificial SequencepCRC203 recombinant MHC class II heterodimer
MFKNIVTPRTPPPGGGSGDSERHFVFQFKGECYFTNGTQRIRSVDRYIYNREEYLRFDSDVGEYRAVTELGRPDPEYYNKQYLEQTRAELDTVCRHNYEGVETHTSLRRLGGEDDIEADHVGVYGTTVYQSPGDIGQYTHEFDGDEWFYVDLDKKETIWMLPEFGQLTSFDPQGGLQNIATGKYTLGILTKRSNSTPATNHHHHHH

SEQ ID NO: 87 - protein, 200 aa, Artificial Sequence
  Description of Artificial SequencepCRC201 recombinant MHC class II heterodimer
MFKNIVTPRTPPPGGGSGDSERHFVFQFKGECYFTNGTQRIRSVDRYIYNREEYLRFDSDVGEYRAVTELGRPDPEYYNKQYLEQTRAELDTVCRHNYEGVETHTSLRRLGGEDDIEADHVGVYGTTVYQSPGDIGQYTHEFDGDEWFYVDLDKKETIWMLPEFGQLTSFDPQGGLQNIATGKYTLGILTKRSNSTPATN

SEQ ID NO: 88 - protein, 217 aa, Artificial Sequence
  Description of Artificial SequencepCRC199 recombinant MHC class II heterodimer
MFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLTSGGGGSEDDIEADHVGVYGTTVYQSPGDIGQYTHEFDGDEWFYVDLDKKETIWMLPEFGQLTSFDPQGGLQNIATGKYTLGILTKRSNSTPATNHHHHHH

SEQ ID NO: 89 - protein, 211 aa, Artificial Sequence
  Description of Artificial SequencepCRC197 recombinant MHC class II heterodimer
MFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLTSGGGGSEDDIEADHVGVYGTTVYQSPGDIGQYTHEFDGDEWFYVDLDKKETIWMLPEFGQLTSFDPQGGLQNIATGKYTLGILTKRSNSTPATN

SEQ ID NO: 90 - protein, 547 aa, Artificial Sequence
  Description of Artificial SequencepCRC188 recombinant MHC class II heterodimer
MALQIPSLLLSAAVVVLMVLSSPGTEGASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 91 - protein, 252 aa, Artificial Sequence
  Description of Artificial SequencepCRC187 recombinant MHC class II heterodimer
MGKKQTAVAFALALLALSMTPAYAFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLGGGGSGGGGSGGGGSGGGGSGGGGSEDDIEADHVGVYGTTVYQSPGDIGQYTHEFDGDEWFYVDLDKKETIWMLPEFGQLTSFDPQGGLQNIATGKYTLGILTKRSNSTPATN

SEQ ID NO: 92 - protein, 353 aa, Artificial Sequence
  Description of Artificial SequencepCB229 recombinant MHC class II heterodimer
MALQIPSLLLSAAVVVLMVLSSPGTEGFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESARSGSADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 93 - protein, 551 aa, Artificial Sequence
  Description of Artificial SequencepCB223 recombinant MHC class II heterodimer
MALQIPSLLLSAAVVVLMVLSSPGTEGEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEPEIPAPMSEGSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 94 - protein, 330 aa, Artificial Sequence
  Description of Artificial SequencepCB212 recombinant MHC class II heterodimer
MALQIPSLLLSAAVVVLMVLSSPGTEGEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEPEIPAPMSEGSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCAA

SEQ ID NO: 95 - protein, 233 aa, Artificial Sequence
  Description of Artificial SequencepCB214 recombinant MHC class II heterodimer
MFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLGGGGSGGGGSGGGGSGGGGSGGGGSEDDIEADHVGVYGTTVYQSPGDIGQYTHEFDGDEWFYVDLDKKETIWMLPEFGQLTSFDPQGGLQNIATGKYTLGILTKRSNSTPATNGTGS

SEQ ID NO: 96 - protein, 559 aa, Artificial Sequence
  Description of Artificial SequencepCB220 recombinant MHC class II heterodimer
MALQIPSLLLSAAVVVLMVLSSPGTEGFKNIVTPRTPPPASGGGGSGGGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWTSGGGGSGGGGSGGGGSGGGGSSSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWASGGGGSGGGGADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 97 - protein, 93 aa, Artificial Sequence
  Description of Artificial Sequencenative IAs beta 1
GDSERHFVFQFKGECYFTNGTQRIRSVDRYIYNREEYLRFDSDVGEYRAVTELGRPDPEYYNKQYLEQTRAELDTVCRHNYEGVETHTSLRRL

SEQ ID NO: 98 - protein, 210 aa, Artificial Sequence
  Description of Artificial SequencemCO608 single chain molecule
MGSERFKNIVTPRTPPPASGGGGSGGGHFVFQFKGECYFTNGTQRIRSVDRYIYNREEYLRFDSDVGEYRAVTELGRPDPEYYNKQYLEQTRAELDTVCRHNYEGVETHTSLRRLTSGGGGSEDDIEADHVGVYGTTVYQSPGDIGQYTHEFDGDEWFYVDLDKKETIWMLPEFGQLTSFDPQGGLQNIATGKYTLGILTKRSNSTPATN

SEQ ID NO: 99 - protein, 206 aa, Artificial Sequence
  Description of Artificial SequencemCO608-A single chain molecule
MFKNIVTPRTPPPASGGGGSGGGHFVFQFKGECYFTNGTQRIRSVDRYIYNREEYLRFDSDVGEYRAVTELGRPDPEYYNKQYLEQTRAELDTVCRHNYEGVETHTSLRRLTSGGGGSEDDIEADHVGVYGTTVYQSPGDIGQYTHEFDGDEWFYVDLDKKETIWMLPEFGQLTSFDPQGGLQNIATGKYTLGILTKRSNSTPATN

SEQ ID NO: 100 - protein, 210 aa, Artificial Sequence
  Description of Artificial SequencemCO608-B single chain molecule
MFKNIVTPRTPPPASGGGGSGGGGSERHFVFQFKGECYFTNGTQRIRSVDRYIYNREEYLRFDSDVGEYRAVTELGRPDPEYYNKQYLEQTRAELDTVCRHNYEGVETHTSLRRLTSGGGGSEDDIEADHVGVYGTTVYQSPGDIGQYTHEFDGDEWFYVDLDKKETIWMLPEFGQLTSFDPQGGLQNIATGKYTLGILTKRSNSTPATN

SEQ ID NO: 101 - protein, 199 aa, Artificial Sequence
  Description of Artificial SequenceCO561 single chain molecule
MGSERFKNIVTPRTPPPGGGSHFVFQFKGECYFTNGTQRIRSVDRYIYNREEYLRFDSDVGEYRAVTELGRPDPEYYNKQYLEQTRAELDTVCRHNYEGVETHTSLRRLGGEDDIEADHVGVYGTTVYQSPGDIGQYTHEFDGDEWFYVDLDKKETIWMLPEFGQLTSFDPQGGLQNIATGKYTLGILTKRSNSTPATN

SEQ ID NO: 102 - protein, 208 aa, Artificial Sequence
  Description of Artificial Sequencegp39-beta1/alpha1 HLA-DR4 CO563 human single chain molecule
MGDTGRSFTLASSETGVGASGGGGSGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 103 - protein, 181 aa, Artificial Sequence
  Description of Artificial Sequence “empty” beta1/alpha1 HLA-DR4 human single chain molecule
MGDTRPRFLEQVKHECHFFNGTERVRFLDRYFYHQEEYVRFDSDVGEYRAVTELGRPDAEYWNSQRDLLEQKRAAVDTYCRHNYGVGESFTVQRRGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 104 - DNA, 36 bp, Artificial Sequence
  Description of Artificial Sequence phosphorylated Primer 1
naccaggaggagagccgcccacgccggtctcgctgg

SEQ ID NO: 105 - DNA, 35 bp, Artificial Sequence
  Description of Artificial Sequence phosphorylated Primer 2
naccacctggatctggggacacccgaccacgtttc

SEQ ID NO: 106 - protein, 4 aa, Artificial Sequence
  Description of Artificial Sequenceupstream linker for CO523 and downstream linker for CO595
GGGG

SEQ ID NO: 107 - protein, 9 aa, Artificial Sequence
  Description of Artificial Sequenceupstream linker for CO563, CO567, CO580, CO581, CO582, CO583, CO584, CO585, CO593, CO595, CO596 and CO597
ASGGGSGGG

SEQ ID NO: 108 - protein, 14 aa, Artificial Sequence
  Description of Artificial Sequencedownstream linker for CO567 and CO586
TSGGGGSGGGGSSS

SEQ ID NO: 109 - protein, 14 aa, Artificial Sequence
  Description of Artificial Sequencedownstream linker for CO580 and CO587
GSPGGGGSGGGPGS

SEQ ID NO: 110 - protein, 10 aa, Artificial Sequence
  Description of Artificial Sequencedownstream linker for CO581 and CO588
GSPPGGPPGS

SEQ ID NO: 111 - protein, 10 aa, Artificial Sequence
  Description of Artificial Sequencedownstream linker for CO582 and CO589
GSPGGGGPGS

SEQ ID NO: 112 - protein, 7 aa, Artificial Sequence
  Description of Artificial Sequencedownstream linker for CO583 and CO590
TSGGGGS

SEQ ID NO: 113 - protein, 7 aa, Artificial Sequence
  Description of Artificial Sequencedownstream linker for CO584 and CO591
SGGSGGS

SEQ ID NO: 114 - protein, 8 aa, Artificial Sequence
  Description of Artificial Sequencedownstream linker for CO585 and CO592
FDAPSPLP

SEQ ID NO: 115 - protein, 7 aa, Artificial Sequence
  Description of Artificial Sequencedownstream linker for CO593 and CO594
VYPEVTV

SEQ ID NO: 116 - protein, 5 aa, Artificial Sequence
  Description of Artificial Sequencedownstream linker for CO596
GGGGS

SEQ ID NO: 117 - protein, 6 aa, Artificial Sequence
  Description of Artificial Sequencedownstream linker for CO597
GGGSGG

SEQ ID NO: 118 - protein, 16 aa, Artificial Sequence
  Description of Artificial Sequencelinker L2 human CD4 binding sequences
RNGQEEKAGVVSTGLI

SEQ ID NO: 119 - protein, 16 aa, Artificial Sequence
  Description of Artificial Sequencelinker L2 human CD4 binding sequences
RNGQETKAGVVSTGLI

SEQ ID NO: 120 - protein, 16 aa, Artificial Sequence
  Description of Artificial Sequencelinker L2 human CD4 binding sequences
YNQQEEKAGGVSTGLI

SEQ ID NO: 121 - protein, 17 aa, Artificial Sequence
  Description of Artificial Sequencelinker L2 human CD4 binding sequences
FRNGQEEKAGVVSTGLI

SEQ ID NO: 122 - protein, 17 aa, Artificial Sequence
  Description of Artificial Sequencelinker L2 human CD4 binding sequences
FRNGQETKAGVVSTGLI

SEQ ID NO: 123 - protein, 17 aa, Artificial Sequence
  Description of Artificial Sequencelinker L2 human CD4 binding sequences
FYNQQEEKAGGVSTGLI

SEQ ID NO: 124 - protein, 16 aa, Artificial Sequence
  Description of Artificial Sequencelinker L2 human CD4 binding sequences
LNGQEEKAGMVSTGLI

SEQ ID NO: 125 - protein, 5 aa, Artificial Sequence
  Description of Artificial Sequencefirst five amino acids after Met in native beta 1 domain, lacking in mouse CO608
GDSER

SEQ ID NO: 126 - protein, 4 aa, Artificial Sequence
  Description of Artificial Sequencefour amino acids after Met in mCO608, lacking in mCO608-A and mCO608-B
GSER

SEQ ID NO: 127 - protein, 4 aa, Artificial Sequence
  Description of Artificial Sequenceupstream linker of mouse CO521
GGGS

SEQ ID NO: 128 - protein, 10 aa, Artificial Sequence
  Description of Artificial Sequenceupstream linker of human CO608
ASGGGGSGGG

SEQ ID NO: 129 - protein, 5 aa, Artificial Sequence
  Description of Artificial Sequencemultimeric complex linker
GGGSG